(19) United States
(12) Patent Application Publication
(10) Pub. No.: US 2020/0054719 A1
(43) Pub. Date: Feb. 20, 2020

(54) E-WE THROMBIN ANALOG AND FIBRINOLYTIC COMBINATION

(71) Applicants: Erik Ian Tucker, Portland, OR (US); Brandon Davis Markway, Portland, OR (US); Michael Nikolaus Wallisch, Portland, OR (US); Nora Green Verbout, Portland, OR (US)

(72) Inventors: Erik Ian Tucker, Portland, OR (US); Brandon Davis Markway, Portland, OR (US); Michael Nikolaus Wallisch, Portland, OR (US); Nora Green Verbout, Portland, OR (US)

(21) Appl. No.: 16/346,651

(22) PCT Filed: Nov. 2, 2017
(86) PCT No.: PCT/US2017/059702

§ 371 Date: May 1, 2019

Related U.S. Application Data
(60) Provisional application No. 62/416,631, filed on Nov. 2, 2016.

(51) Int. Cl. A61K 38/48; A61K 38/49; A61P 9/10; A61P 7/02

(52) U.S. Cl. CPC A61K 38/4833; A61P 7/02; A61P 9/10; A61K 38/49

(57) ABSTRACT

According to the invention, a novel combination composition and method of treatment for thrombotic disorders, e.g., STEMI in ACS patients, is disclosed. The present invention relates to thrombin analogs, e.g., WE and E-WE thrombin analogs, in combination with fibrinolytics, e.g., tPA. In particular, E-WE thrombin analog and fibrinolytic combination therapy for inhibition of thrombin mediated TAFi activation and acceleration of tPA induced thrombolysis with E-WE thrombin. The present invention also relates to methods of treating a subject having a thrombotic or thromboembolic disorder by delivering the novel composition comprised of at least one antithrombotic thrombin analog and at least one fibrinolytic agent to the subject.

### RELATED APPLICATIONS

This application claims the benefit of priority from U.S. provisional patent application No. 62/416,631, filed on 2 Nov. 2016, the disclosure of which is hereby incorporated by reference in its entirety.

### GOVERNMENT SUPPORT

The present invention was made, at least in part, with governmental support pursuant to SBIR grant R44HL117589 awarded by the National institutes of Health. Accordingly, the government has certain rights to the invention.

### SEQUENCE LISTING

The instant application contains a Sequence Listing which has been filed electronically in ASCII format and is hereby incorporated by reference in its entirety. Said ASCII copy, created on Nov. 1, 2017, is named ARB001PCT_SL.txt and is 75,765 bytes in size.

### FIELD OF INVENTION

The present invention relates generally to compositions useful for the treatment of acute thrombotic emergencies, and methods of using a therapeutic composition comprising thrombin analogs as fibrinolysis enhancing agents in combination with fibrinolytics. More specifically, the present invention is directed to methods for enhancing fibrinolysis with antithrombotic human thrombin analogs, e.g., WE and E-WE thrombin, in combination with fibrinolytics, e.g., tissue plasminogen activator (“tPA”), for antithrombotic intervention.

### BACKGROUND OF THE INVENTION

Acute myocardial infarction (AMI) is a medical emergency that affects approximately 1 million Americans each year and remains a leading cause of death in the United States. The most prevalent cause of AMI is myocardial ischemia due to rapid thrombosis progression within coronary arteries. This can ultimately lead to complete occlusion, also known as ST-segment elevation myocardial infarction (STEMI), which occurs in roughly 30% of patients. Successful STEMI treatment includes rapid reperfusion of the ischemic myocardium using methods such as percutaneous transluminal coronary angioplasty (PTCA) or pharmacological thrombolysis (fibrinolysis), which are generally combined with sustained antithrombotic therapy to prevent further thrombotic progression and subsequent ischemia into the functioning myocardium. Rapid reperfusion therapy results in smaller infarct size, minimized myocardial damage, preserved left ventricular function, and reduced morbidity and mortality.

Current ACC/AHA guidelines and recent analyses from the Global Registry of Acute Coronary Events demonstrate a preference for percutaneous coronary intervention (PCI) over pharmacological thrombolysis, but only 25% of U.S. hospitals are PCI-capable, and consequently 60% to70% of STEMI patients initially present at non-PCI capable facilities. For these patients, rapid fibrinolysis treatment is recommended, even if timely transfer to a PCI-capable facility can be performed. However, myocardial reperfusion by way of thrombolytic therapies has several limitations, the foremost being a lack of thrombosis specificity and an elevated risk for bleeding. Current fibrinolytics and antithrombotic treatments, such as tissue plasminogen activator (tPA) and heparin, disable hemostasis, thereby increasing the risk of life-threatening bleeding. As a result, delayed treatment often occurs, with up to 30% receiving no reperfusion treatment at all. These safety and logistical limitations of delivering timely reperfusion therapy to STEMI patients undoubtedly cost many lives. Accordingly, there is a significant medical need for safe antithrombotic and thrombolytic treatments that can be used alone or can be combined with currently approved therapies for improved efficacy and more rapid reperfusion without increased bleeding risks.

The development of novel therapeutic composition or combination therapy comprising antithrombotic and thrombolytic aspects would provide a much needed treatment for acute thrombotic emergencies. The present invention solves this need and overcomes each of the shortcomings described hereinabove. The potential clinical benefit of a novel composition for combination therapy, e.g., E. coli derived, non-glycosylated, WE thrombin (E-WE thrombin) and tPA, in STEMI patients includes aspects such as reduced time for successful and adequate myocardial reperfusion. Taken together, the novel combination and its unique features result in a drug candidate composition and method of use that may have significant impact on treatment outcomes for STEMI and other acute coronary syndrome (ACS) patients.

### SUMMARY OF THE INVENTION

The present invention overcomes the existing drawbacks and prior art by providing novel thrombin analog and fibrinolytic combination composition and method of treatment for the inhibition of thrombin mediated thrombin-activatable fibrinolysis inhibitor (TAFI) activation and acceleration of tPA induced thrombolysis. Advantages of the present invention include, for example, a novel combination treatment for STEMI in ACS patients that includes thrombin analog and fibrinolytic combination composition for inhibition of thrombin mediated TAFI activation and acceleration of thrombolysis.

In some embodiments, the invention comprises, at east one antithrombotic thrombin analog and at least one fibrinolytic agent. In some embodiments, the thrombin analog may be a fully glycosylated WE thrombin analog. In some embodiments, the thrombin analog may be a non-glycosylated E-WE thrombin analog.

In some aspects, the present invention contemplates an animal cell derived (expressed) recombinant WE thrombin and corresponding precursors (SEQ ID NOS:2, 10, 11, 19), for example, tPA induced thrombolysis with WE thrombin and tPA. In some aspects, the present invention contemplates bacterial cell derived (expressed) recombinant E-WE thrombin and corresponding precursors (SEQ ID NOS:1, 5, 6, 15, 20, 22), for example, tPA induced thrombolysis with E-WE thrombin and tPA.

In some embodiments, the combination composition may include at least one thrombin analog that comprises the amino acid sequence set forth in SEQ ID NO:1, 2, or 22. In some embodiments, these thrombin analog may comprise thrombin mutants that either partially or fully disable procoagulant activity and retain a portion or all of the protein C activating ability of wild type thrombin and corresponding precursors (SEQ ID NOS:3, 4, 7, 8, 9, 17).

In some embodiments, the thrombin analog may comprise an ecarin activatable analog (SEQ ID NOS: 5, 6, 7, 8, 15, 17, 20) and/or comprise a cleavage site (SEQ ID NOS: 12, 13, 14, 16, 18, 21, 22, 23, 24).

In some embodiments, at least one fibrinolytic agent may be selected from the group consisting of scuPA, tPA, uPA, tcuPA, streptokinase, rt-PA, alteplase, rt-PA derivatives, reteplase, lanoteplase, TNK-re-PA, anisoylated plasminogen streptokinase complex, anistreplase, streptokinase derivative, and variations and combinations thereof. In some embodiments, at least one fibrinolytic agent may be tPA.

In some embodiments, the combination composition treatment may include at least one thrombin analog wherein the analog is comprised of the amino acid sequence as set forth in SEQ ID NO: 1, 2, or 22, and the at least one fibrinolytic agent may be selected from the group consisting of scuPA, tPA, uPA, tcuPA, streptokinase, rt-PA, alteplase, rt-PA derivatives, reteplase, lanoteplase, TNK-re-PA, anisoylated plasminogen streptokinase complex, anistreplase, streptokinase derivative and combinations thereof.

In some embodiments, a pharmaceutically acceptable composition for promoting thrombus dissolution in a subject may comprise: a thrombin analog, wherein the analog is comprised of the amino acid sequence as set forth in SEQ ID NO: 1 2, or 22; and a fibrinolytic agent, wherein the agent is tPA.

Some embodiments comprise methods for treating subjects having a thrombotic or thromboembolic disorder and may comprise administering a composition according to any combination of thrombin analogs and fibrinolytics disclosed. Some embodiments may comprise a method of treatment by administering a composition comprising: a thrombin analog, wherein the analog is comprised of the amino acid sequence as set forth in SEQ ID NO: 1, 2, or 22; and, a fibrinolytic agent, wherein the agent is tPA.

Some embodiments may comprise a method of fibrinolytic therapy in a subject in need thereof, wherein the method comprises administering to the subject an effective dosage of a thrombolytic agent prior to, subsequent to, or concurrently with administering an effective dosage of an E-WE or WE thrombin analog. In some embodiments, the E-WE thrombin analog is the analog set forth in SEQ ID NO:1. In some embodiments, the E-WE thrombin analog is the analog set forth in SEQ ID NO:22. In some embodiments, the WE thrombin analog is the analog set forth in SEC) ID NO:2.

In some embodiments, the invention further comprises at least one pharmaceutically acceptable carrier, diluent, excipient, wetting agent, emulsifier, buffer, adjuvant, viscosity additive, preservative, acid, base, salt, sugar, and variations and combinations thereof.

In some embodiments, the invention comprises kits comprised of elements for practicing the methods and/or the compositions set forth herein. In some embodiments, the contemplated kit is comprised of a pharmaceutically acceptable composition comprising at least one thrombin analog and at least one fibrinolytic agent, and packaging comprising instructions for administration of the composition to a subject.

Broadly, the present invention contemplates a combination composition treatment for STEMI and methods of inhibiting thrombus formation and/or lysing formed thrombi in an animal or human subject by delivering the combination composition. The present invention also relates to methods of treating a subject having a thrombotic or thromboembolic disorder by delivering the combination composition treatment comprised of an antithrombotic thrombin analog and fibrinolytic to the subject. When practiced as disclosed herein, the present invention provides a novel, effective, ACS patient therapeutic composition and treatment that reduces time for successful and adequate myocardial reperfusion, and thus, is useful as disclosed herein but is not intended to be limited to these uses.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIGS. 1A-C illustrate E-WE thrombin reduction of myocardial infarct size following experimental ischemia. FIG. 1A depicts an experimental protocol and time course for in vivo model of myocardial ischemia-reperfusion; FIG. 1B shows the risk size for each group evaluated at 2 and 24 hr expressed as the percent of total heart volume; and FIG. 1C shows infarct size for each group evaluated at 2 and 24 hr expressed as percentage of area at risk.

FIGS. 2A-2E illustrate E-WE thrombin improvement of cardiomyocyte survival following experimental ischemic-reperfusion. FIG. 2A depicts an experimental timeline and treatments for ex vivo model of oxygen glucose depletion/re-oxygenation and glucose repletion (OGD/RGR); FIG. 2B shows concentration dependent improved cell survival with pretreatment with E-WE thrombin; FIG. 2C shows protocol adaptation to serum-free media with comparable outcome; FIG. 2D shows adapted protocol conferred E-WE thrombin dependent cardioprotective effects; and FIG. 2E shows confirmation of E-WE-mediated cardioprotection.

FIGS. 3A-3B illustrate E-WE thrombin dose dependently reduces thrombus growth in collagen-coated grafts. FIG. 3A shows platelet deposition in graft head section; and FIG. 3B shows platelet deposition in graft tail section.

FIG. 4 illustrates E-WE thrombin dose dependently reduces fibrin content in collagen-coated grafts and enhances tPA mediated fibrinolysis.

FIGS. 5A-5B illustrate E-WE thrombin competitively inhibits TAFI activation by thrombin in vitro. FIG. 5A shows activated TAFI produced over time by wild-type (WT) and E-WE thrombin in the presence or absence of thrombomodulin (TM); and FIG. 5B shows E-WE thrombin dose dependent inhibition of TAFI activation.

FIGS. 6A-6B illustrate E-WE thrombin accelerates clot lysis induced by tPA in baboon plasma in vitro. FIG. 6A shows clot lysis time plotted as amount of tPA versus lysis time in seconds; and FIG. 6B shows clot lysis plotted as tPA v. lysis time as percent baseline.

FIG. 7 illustrates the amino acid sequence of E-WE thrombin (SEQ ID NO: 1). A-chain is depicted in bold, and W215A and E217 mutations are underlined.

### DETAILED DESCRIPTION OF THE INVENTION

The embodiments of the present invention described herein provide exemplary embodiments only, and are not intended to be exhaustive, limit the scope, applicability or configuration of the disclosure. Rather, the description of the exemplary embodiments provides those skilled in the art with an enabling description for implementing one or more exemplary embodiments. It is understood by those skilled in the art that various changes may be made in the function and arrangement of elements without departing from the spirit and scope of the invention as set forth in the appended claims.

Unless specifically noted, it is intended that the words and phrases in the specification and the claims be given their plain ordinary and accustomed meaning to those of ordinary skill in the applicable arts. Accordingly, various implementations may be very broadly adopted and applicable. As used herein, all defined terms include analogous and partially analogous terms that those skilled in the art would refer to as analogous or equivalent, or at least partially analogous or the like.

As used herein, “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (for example, X₁ and X₂) as well as a combination of elements selected from two or more classes (for example, Y₁ and Zₒ).

The use of the terms “a” and “an” and “the” and similar referents in the context of describing the invention (especially in the context of the following claims) is to be construed to cover both the singular and the plural, unless otherwise indicated herein or clearly contradicted by context. As such, the terms “a” (or “an”), “one or more” and “at least one” can be used interchangeably. The terms “comprising,” “having,” “including,” and “containing” are to be construed as open-ended terms (i.e., meaning “including, but not limited to,”) unless otherwise noted. As such, the terms “comprising,” “including,” “containing,” and “having” can be used interchangeably.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The term “means” as used herein shall be given its broadest possible interpretation in accordance with 35 U.S.C. Section 112. Accordingly, a claim incorporating the term “means” shall cover all structures, materials, or acts set forth herein, and all of the equivalents thereof. Further, the structures, materials, or acts and the equivalents thereof shall include all those described in the summary, brief description of the drawings, detailed description, abstract, and claims themselves.

It should be understood that every maximum numerical limitation given throughout the present disclosure is deemed to include each and every lower numerical limitation as an alternative, as if such lower numerical limitations were expressly written herein. Every minimum numerical limitation given throughout the present disclosure is deemed to include each and every higher numerical limitation as an alternative, as if such higher numerical limitations were expressly written herein. Every numerical range given throughout the present disclosure is deemed to include each and every narrower numerical range that falls within such broader numerical range, as if such narrower numerical ranges were all expressly written herein. Recitation of ranges of values herein are merely intended to serve as a shorthand method of referring individually to each separate value falling within the range, unless otherwise indicated herein, and each separate value is incorporated into the specification as if it were individually recited herein.

All amino acid residues identified herein are in the natural L-configuration. In keeping with standard polypeptide nomenclature, IUPAC-IUB Commission on Biochemical Nomenclature (1969) J. Biol. Chem. 243:3557-3559, abbreviations for amino acid residues are as shown in the following Table of Correspondence:

TABLE 1
Table of Amino Acid Symbol Correspondence

| 1-letter | 3-letter | Amino acid |
|---|---|---|
| A | Ala | Alanine |
| C | Cys | Cysteine |
| D | Asp | Aspartic Acid |
| E | Glu | Glutamic Acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| Y | Tyr | Tyrosine |

Described herein are novel therapeutic compositions and methods of use, comprised of at least one hemostatically safe antithrombotic for the treatment of STEMI e.g., WE and E-WE thrombin analogs, in combination with at least one fibrinolytic agent, such as tPA, for enhanced thrombolysis without added hemostatic impairment. WE is a double mutant thrombin variant expressed in mammalian cells. E-WE thrombin is an E. coli expressed WE double mutant thrombin variant. Both thrombins are thrombomodulin-dependent thrombin analogs that generate endogenous activated protein C (APC) on intravascular surfaces. In vivo, WE and E-WE thrombin inhibit experimental thrombus propagation without systemic anticoagulation or hemostasis impairment. In vitro and in vivo data suggest that E-WE thrombin unexpectedly promotes and accelerates tPA-mediated fibrinolysis.

Thrombosis is caused by fibrin and platelet deposits that occlude blood vessels. The activation of naturally occurring physiologic systems leading to the production of endogenous therapeutic proteins can be efficacious and economical. For example, plasminogen activators are valuable in the systemic treatment of thrombosis. Wild type (WT) thrombin is an antithrombotic enzyme that is capable of binding to thrombomodulin and generating endogenous APC. However, fibrin formation and platelet activation therefrom have potentially adverse side effects, and thrombin not complexed with thrombomodulin can cause significant intravascular coagulation.

“Thrombin” as used herein, (SEQ ID NO:3) refers to a multifunctional prothrombin derived enzyme, a serine protease, which in humans is encoded by the F2 gene. Prothrombin is cleaved to form thrombin in the coagulation cascade, and in turn, thrombin converts soluble fibrinogen into insoluble fibrin and catalyzes other coagulation related reactions. Thus, thrombin acts as a procoagulation agent by the proteolytic cleavage of fibrinogen to fibrin, activates clotting factors V, VIII, XI, and XIII, and cleaves the platelet thrombin receptor PAR-1 leading to platelet activation. However, multiple antithrombotic mechanisms limit thrombin generation and activity.

When thrombin binds to thrombomodulin (TM), an integral membrane protein on vascular endothelial cells, thrombin undergoes a conformational change and loses it procoagulation activity. It then acquires the ability to convert protein C (PC) to activated protein C (APC). APC acts as a potent anticoagulant by inactivating active FV (FVa) and FVIII (FVIIIa), two essential cofactors in the clotting cascade. APC also inactivates plasminogen activator inhibitor-1 (PAI-1), the major physiologic inhibitor of tissue plasminogen activator (tPA), thus potentiating normal fibrinolysis.

The double thrombin mutant referred to as W215A/E217A (WE thrombin (SEQ ID NO: 2)) is constructed by combining the two single mutations W215A and E217A in the thrombin molecule (Cantwell, (2000) J. Biol. Chem. 275:39827-39830). W215A and E217A refer to amino acid residue positions in the thrombin ammo acid residue sequence using the position numbers as described in Bode et al (1989) EMBO. J., 8:3467-3475), that correspond to sequential amino acid residue positions 263 and 265 from the N-terminus of thrombin, respectively. WE thrombin is known to exhibit antithrombotic activity in vivo, without any direct anticoagulant activity. Its antithrombotic effect has been shown in non-human primates to be more efficacious than the direct administration of activated protein C, and safer than the administration of low molecular weight heparins.

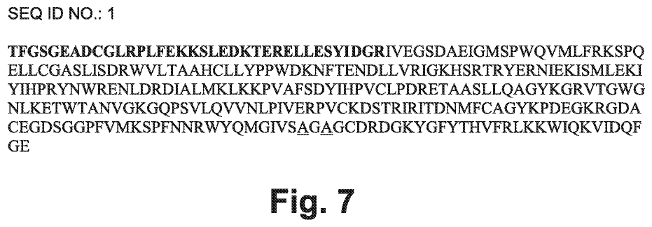

E-WE thrombin (SEQ ID NO: 1) is an Escherichia coli culture-derived or—expressed WE thrombin. E-WE thrombin is a proprietary, first-in-class drug candidate disclosed in U.S. Pat. Nos. 6,706,512, 7,223,583, and 8,940,297, the amino acid sequence of which is shown in FIG. 7. It is structurally and functionally similar to wild type (WT) thrombin and, like thrombin, is a potent activator of protein C, the endogenous enzyme possessing powerful and essential antithrombotic and cytoprotective activities. Recombinant E-WE thrombin prepared from a bacteria-expressed precursor is safer, e.g., has less activity towards procoagulant substrates, and has similar or possibly slightly enhanced anticoagulant (anti-thrombotic) therapeutic effects as glycosylated WE thrombin expressed in a mammalian cell line from the same DNA coding sequence. Thrombin analogs, variants, and fragments thereof (SEQ ID NOS:3, 4, 7, 8, 9, 17), preferably WE thrombin analogs, variants, and fragments thereof (SEQ ID NOS:2, 10, 11, 19), and more preferably, E-WE thrombin analogs, variants, and fragments thereof (SEQ ID NOS.1, 5, 6, 15, 20, 22). are the preferred analogs of thrombin useful in the present invention, each of which is set forth in U.S. Pat. No. 8,940,297, specifically incorporated herein by reference in its entirety. More specifically, WE and E-WE thrombin analogs most useful in the present invention have substantially reduced procoagulant activity, a compromised platelet activation activity, and the capability to activate protein C. The thrombin analogs are also practically devoid of activity toward fibrinogen and the platelet receptor PAR-1.

Two distinct amino acid numbering systems are in use for thrombin in addition to the DNA-based system of Degen et al. (Biochemistry (1993) 22:2087) and may be utilized herein. One is based on alignment with chymotrypsinogen as described in Bode et al. (EMBO. J. (1989) 8:3467-3475), and a second, the Sadler numbering scheme, in which the B chain of thrombin commences with I1 and extends to E259, while the A chain is designated with “a” postscripts as in T1a to R36a.

Analogs that carry WT or WE thrombin amino acid residue sequence, and precursors thereto, are contemplated for use with this invention in various embodiments to improve therapeutic efficacy and safety include, for example, E-WE thrombin (SEQ ID NO:1), WE thrombin (SEQ ID NO:2), thrombin (SEQ ID NO:3), preprothrombin (SEQ ID NO:4), ecarin-activatable E-WE preprothrombin (SEQ ID NO:5), ecarin-activatable E-WE prethrombin-2 (SEQ ID NO:6), ecarin-activatable preprothrombin (SEQ ID NO:7), ecarin-activatable prethrombin-2 (SEQ ID NO:8), ecarin-activatable Δ146-149e prethrombin-2 (SEQ ID NO:9), WE preprothrombin (SEQ ID NO:10), WE prethrombin-2 (SEQ ID NO:11), ecarin-activatable E-WE thrombin precursor A (SEQ ID NO:15), ecarin-activatable thrombin precursor A (SEQ ID NO:17), WE prothrombin (SEQ ID NO:19), ecarin-activatable E-WE prothrombin SEQ ID NO:20), E-WE thrombin with ecarin site (SEQ ID NO:22), and variations and combinations thereof, and may further be combined with cleavage sites, for example, SEQ ID NOS:12, 13, 14, 16, 18, 21, 23, 24.

The thrombin analogs contemplated by the present invention are useful agents suitable for administering in combination compositions to a subject. Contemplated thrombin precursors useful in preparing WE and E-WE thrombin for use in combination with and to enhance the performance of fibrinolytics need not be well known. They may be any thrombin precursor, fusion peptide, or polypeptide known or yet to be discovered or created. The preferred antithrombotic thrombin analogs described herein, WE thrombin or E-WE thrombin, may be combined with a fibrinolytic for therapeutic use, e.g., enhancing hemostasis, or treating and preventing thrombosis. WE and E-WE thrombin are 2-chain polypeptides and cannot be prepared from a single polypeptide chain without post expression processing. As such, the polynucleotides encode each of the WE and E-WE ecarin site-containing precursors, e.g., WE and E-WE preprothrombin, WE and E-WE prothrombin, WE and E-WE prethrombin-1, and WE and E-WE prethrombin-2, and can be used to express a polypeptide precursor that can be further processed, e.g., with ecarin, to provide a WE or E-WE thrombin for use in a contemplated composition or method as disclosed herein. Thrombin precursors that have enzymatic activity but must be acted upon to form thrombin are deemed active precursors of thrombin herein. The present invention, thus, further contemplates that the precursors may be administered to a subject to be cleaved in vivo in order to deliver the corresponding thrombin to the subject, or may be cleaved ex vivo prior to administration to a subject. For example, the snake-venom derived enzyme ecarin may be used to cleave prothrombin to produce thrombin.

Fibrinolytics comprises a group of drugs that are capable of breaking down fibrin. Fibrin is the protein that is a primary constituent of a thrombus. Fibrinolytics are useful to disperse a thrombus and may be used for the immediate treatment of, e.g., acute myocardial infarction, deep vein thrombosis, pulmonary embolism, and the like. There are three major classes of fibrinolytic drugs: tissue plasminogen activator (tPA), streptokinase (SK), and urokinase (UK). Drugs in all three classes have the ability to effectively dissolve thrombi.

tPA and derivatives are the most commonly used thrombolytic drugs, especially for coronary and cerebral vascular thrombi because of their relative selectivity for activating fibrin-bound plasminogen. tPA is therefore used, for example, in acute myocardial infarction, cerebrovascular thrombotic stroke, and pulmonary embolism.

SK and derivatives are not a protease and have no enzymatic activity, however, form a complex with plasminogen that releases plasmin. Unlike tPA, SK does not bind preferentially to clot-associated fibrin and therefore binds equally to circulating and non-circulating plasminogen. UK and derivatives are sometimes referred to as urinary-type plasminogen activator (uPA) because it is formed by kidneys and found in urine.

Examples of fibrinolytics that may be useful in the present invention include, for example, scuPA, tPA, uPA, tcuPA, streptokinase, rtPA, alteplase, rtPA derivatives, reteplase, lanoteplase, TNK-re-PA, anisoylated plasminogen streptokinase complex, anistreplase, streptokinase derivative, and variations and combinations thereof.

A preferred fibrinolytic agent of the present invention, tissue plasminogen activator (tPA), is a protein involved in the breakdown of blood clots (thrombi). It is a serine protease naturally found on endothelial cells that line the blood vessels. It catalyzes the conversion of plasminogen to plasmin, the major enzyme responsible for clot breakdown. Because it works on the clotting system, tPA or recombinant tPA (rtPA) may be used to treat diseases that feature blood clots, such as pulmonary embolism myocardial infarction, and stroke, in a medical treatment called thrombolysis.

As used herein, the term “thrombus” or “thrombi” refers to a coagulated intravascular mass formed from the components of blood that results from a pathological condition of a subject, i.e., animal or human. A thrombus comprises a cross-linked and concentrated mesh of fibrin monomers (fibrin polymer) that entrap platelets and other blood cells. The term “coagulation” refers to the process of polymerization of fibrin monomers, resulting in the transformation of blood or plasma from liquid to gel. “Thrombosis,” as used herein, refers to the pathological formation of a blood clot, or thrombus, which results in restricted or blocked blood flow, with or without clinical symptoms. Thrombotic diseases may include, e.g., ischemic stroke, myocardial infarction, deep vein thrombosis, disseminated intravascular coagulation in sepsis, and the like. “Thromboembolism” refers to a blockage of a blood vessel due to the detachment of a thrombus from its site of origin and translocation to another site in the same or different vessel.

“Thrombotic or thromboembolic disorders” refer to disorders which occur both in the arterial and in the venous vasculature. In particular, disorders in the coronary arteries of the heart, such as acute coronary syndrome (ACS), myocardial infarction with ST segment elevation (STEMI) or without ST segment elevation (non-STEMI), stable angina pectoris, unstable angina pectoris, reocclusions or restenoses after coronary interventions such as angioplasty, stent implantation, or aortocoronary bypass, but also thrombotic or thromboembolic disorders in further vessels leading to peripheral arterial occlusive disorders, pulmonary embolisms, venous thromboembolisms, venous thromboses, in particular in deep leg veins and kidney veins, transitory ischemic attacks, thrombotic stroke, and thromboembolic stroke.

The terms “composition” or “compositions” as used herein refer to pharmaceutically (and physiologically) acceptable therapeutic agents, drugs, substances, or combinations thereof, used on or in the body for the prevention, diagnosis, mitigation, alleviation, treatment, or cure of disorder or disease in human or animal subject, wherein the agents, drugs, substances, or combinations thereof that may be administered in the form of a single compound or formulation, or individually and simultaneously, or individually and sequentially. It is envisioned that compositions of the present invention may be preferentially administered intravenously and/or directly into the thrombus, alone, in combination simultaneously, or in combination sequentially, and/or with other treatments or therapies. The term “subject” as used herein refers to a mammal in need of treatment and to which a pharmaceutical composition containing a contemplated composition is administered. Subjects may be primates, e.g., human, ape, monkey, or laboratory animals, e.g., rat, mouse, rabbit, or companion animals, e.g., dog, cat, horse, or a farm animal, e.g., cow, sheep, lamb, pig, goat, llama, or the like.

The compositions of the present invention are contemplated to contain an effective amount of at least one thrombin analog, and an effective amount of at least one fibrinolytic, one or both of which may be dissolved or dispersed in a compatible pharmaceutically acceptable carrier. Alternatively, the compositions of the present invention may be a liquid, for example, housed in a prefilled syringe or other acceptable or appropriate delivery system, may be a lyophilized product ready to receive carrier (e.g., diluent), may be individual components that are co-administered simultaneously or sequentially, or any combination thereof. The phrase “pharmaceutically acceptable” refers to molecular entities and compositions that typically do not produce an allergic or similar reaction, or the like, when administered to a subject. The amount of the present composition utilized in each administration is referred to as an antithrombotic effective amount and can vary widely, depending, inter alia, upon the subject to which the composition is administered and the severity of the disease state being treated. Standard pharmaceutical texts may be consulted to prepare suitable preparations of the compositions of the present invention without undue experimentation. Alternatively, contemplated embodiments of the present invention include kits comprising instruction for the preparation and/or use of the contemplated compositions of the present invention.

It is contemplated that pharmaceutical compositions or therapeutic treatment methods of the present invention comprising a combination of at least one thrombin analog, e.g., WE or E-WE thrombin, and at least one fibrinolytic agent, e.g., tPA, may be administered in effective dosages, routes of administration, and by other techniques well know to those skilled in the art, medical and/or veterinary by taking into consideration factors such as age, sex, weight, species, and condition of the subject. For example, E-WE thrombin dosages generally may range from about 0.1 to 100 μg/kg, 0.1 to 10 μg/kg, 0.5 to 5.0 μg/kg, or 0.10 to 1.0 μg/kg body weight in combination with a calculated recommended dosage of tPA, for direct injection into the thrombus (consistent with specific product instructions and/or skill in the art). Dosages may be administered as a bolus or over a sustained period, one or multiple administrations, as determined by condition and need of a subject. The composition of the present invention may be administered to a subject as is necessary to achieve the degree of activity desired. The composition may be formed in situ (in vivo or in vitro) or within the body of the subject. Formulation of pharmaceutical compositions is discussed, for example, in Hoover. John E., Remington's Pharmaceutical Sciences, Mack Publishing Co., Easton, Pa.; and Liberman, H. A. and Lachman, L., Eds., Pharmaceutical Dosage Forms, Marcel Decker, New York, N.Y.

Effective routes of administration may be via any route that delivers a safe and effective dose of a composition of the present invention to the subject, for example, parenterally. The term “parenteral” as used herein includes, e.g., intravenous, subcutaneous, intramuscular, intrasternal, or infusion. Additional routes of administration may include, for example, intraperitoneal, intrathecal, intraarticular, intrapulmonary, intrapleural, percutaneous, transmucosal, oral, gastrointestinal, and intraocular.

To enhance the contemplated compositions, the compositions may further comprise pharmaceutically acceptable and suitable carriers, diluents, excipients, wetting agents, emulsifying agents, buffering agents, adjuvant, viscosity additives, preservatives, acids, bases, salts, sugars, and the like, and variations and combinations thereof, both well known in the art and yet to be developed. For example, injectable compositions are typically sterile aqueous preparations or suspensions in nontoxic pharmaceutically acceptable diluent or solvent. Solvents may include, for example, water, Ringer's solution, isotonic sodium chloride solution, and phosphate-buffered saline. Sterile solutions may be prepared by dissolving the active components of the composition in the desired solvent system. Suitability of carriers depends on the intended use, and may include, e.g., saline, PBS, dextrose, glycerol, ethanol, or the like, and variations and combinations thereof.

It has been shown that low-dose thrombin (<0.3 μg/kg/hr) is antithrombotic in primates through the activation of protein C, but the therapeutic window of thrombin is far too narrow for its safe clinical utilization. Subsequent alanine scanning studies identified key residues involved in thrombin substrate specificity, leading to the rational design of thrombin analogs with severely impaired procoagulant activity. One particular thrombin mutant, W217A/E217A (WE thrombin), profoundly reduces catalytic activity towards all tested prothrombotic substrates, including fibrinogen and platelet protease-activated receptor 1 (PAR1), but retains activity towards protein C when in complex with the receptor thrombomodulin (TM). In baboons, low doses of WE thrombin or E-WE thrombin, produce considerable antithrombotic effects, but without any measurable hemostatic impairment.

Activated protein C (APC) anticoagulates blood by rapid enzymatic degradation of coagulation factors Va and VIIIa. It is a potent antithrombotic enzyme in primates and also activates cytoprotective mechanisms through endothelial PAR1-mediated signaling. However, systemic APC administration has the potential to impair hemostasis since APC that is not bound to receptors remains active in the fluid phase of blood, and indeed, systemic APC treatment has been shown to increase bleeding. Large systemic doses of APC are also required to deliver any antithrombotic effects due to its rapid inactivation by protease inhibitors in blood, and these high doses may also contribute to bleeding. Since E-WE thrombin activates only a small amount of receptor-bound protein C, the beneficial effects of APC can be targeted more efficiently to the site of thrombus development. As has been shown, about a 50-fold higher dose of APC must be administered to approach the antithrombotic efficacy of 2 μg/kg of E-WE thrombin.

This mechanistic concept of WE and E-WE thrombin as a protein C activator is analogous to tPA acting as a plasminogen activator. Data suggest that WE and E-WE thrombin may be effectively delivered to a thrombus by circulating platelets (via GPIb and TM) and leukocytes that are actively recruited to the growing thrombus, thereby concentrating the enzyme and generating antithrombotic APC in situ. Furthermore, WE and E-WE thrombin are likely active at all vessel wall locations where the endothelium expresses both TM and endothelial protein C receptors (EPCR), limiting the potential for distal thromboembolism growth and blood vessel obstruction. The novel combination of the present invention shifts the antithrombotic treatment paradigm by demonstrating pharmacological thrombosis targeting by WE and E-WE thrombin, and acceleration of tPA-induced fibrinolysis by subsequent inhibition of TAFI activation. Further, in combination, WE or E-WE thrombin and tPA act as anticoagulant and profibrinolytic agents without enhancing hemostasis impairment beyond the effects of tPA alone. Thus, in addition to being a potent protein C activator, WE and E-WE thrombin, when co-administered with a fibrinolytic such as tPA, promote and accelerate tPA-induced fibrinolysis by inhibiting TAFI activation.

TAFI circulates as a plasminogen-bound zymogen and is activated by the thrombin/thrombomodulin complex. Once activated, TAFI inhibits plasmin-induced fibrinolysis by cleaving the C-terminal residues from fibrin that are important for binding and activation of plasminogen. TAFI activation during thrombolysis may limit the effectiveness of tissue plasminogen activator (tPA) treatment. Therefore, a combination composition of or treatment with an antithrombotic human thrombin analog, e.g., E-WE or WE thrombin, which is a selective protein C activator, co-administered with tPA promotes tPA-induced fibrinolysis. When both E-WE thrombin and tPA are co-administered into plasma clots, lysis can be accelerated by up to 74% when compared to tPA alone.

The enhancement and acceleration of fibrinolysis with antithrombotic human thrombin analogs in combination with fibrinolytics for antithrombotic intervention and treatment of thrombotic disorders is unexpected. More specifically, the synergistic combination treatment of E-WE thrombin and tPA inhibits TAFI activation and concurrently enhances tPA-induced clot lysis in a concentration-dependent manner. It is reasonably expected for WE thrombin to possess the same or similar performance properties as E-WE because the difference between the two thrombin analogs is expression in mammalian cells versus bacterial cells, respectively. Thus, the expectation is that both thrombins and their precursors perform comparably. This novel combination treatment may safely and effectively decreases the time to myocardial reperfusion in STEMI subjects, which on average requires around 60 min to achieve adequate fibrinolysis and reperfusion (TIMI flow grade-3). The combination of WE or E-WE thrombin and tPA therefore addresses the unmet medical need for safe and effective antithrombotic therapy having a direct impact on the treatment and outcome of heart attack and other life-threatening thrombotic medical emergencies.

In one aspect of the present invention, the composition is comprised of at least one thrombin analog, preferably a WE thrombin analog, and more preferably, an E-WE thrombin analog, and at least one fibrinolytic agent. In another aspect of the present invention, the composition is comprised of the E-WE or WE thrombin analog having the amino acid sequence as set forth in SEQ ID NO:1 or 22, or SEQ ID NO:2, respectively, and at least one fibrinolytic agent, preferably tPA. The contemplated thrombin analogs may either partially or fully disable the procoagulant activity, but retain a portion or all of the protein C activating ability of wild type thrombin.

Another aspect of the present invention provides a method of fibrinolytic therapy in a subject in need thereof, the method comprising the steps of administering to the subject a pharmaceutically acceptable therapeutic composition of the present invention comprising an effective dosage of a thrombolytic agent prior to, subsequent to, or concurrently with, an effective dosage of a thrombin analog, preferably an E-WE or WE thrombin analog, and more preferably the E-WE or WE thrombin analog comprising the amino acid sequence set forth in SEQ ID NO: 1 or 22, or SEQ ID NO:2, respectively.

Another aspect of the present invention is a kit comprising a composition of the present invention, and packaging comprising instructions for preparing and/or administering the composition to a subject, e.g., to induce antithrombotic activity in a subject. The kit may further comprise at least one additional pharmaceutically acceptable element, a delivery system, and/or instructions for use thereof.

### EXAMPLE 1

### Evaluation of E-WE Thrombin Treatment in a Mouse Model of Acute Myocardial Ischemia

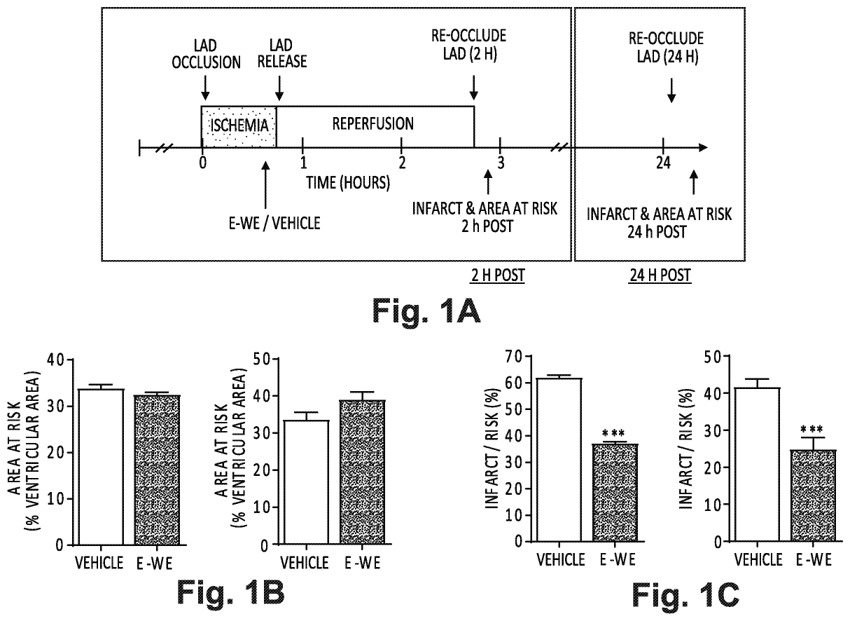

A reduction in infarct size by treatment with E-WE thrombin was demonstrated in this study (FIGS. 1A-C) by inducing transient ischemia by reversibly ligating the left anterior descending coronary artery (LAD). In FIG. 1A, experimental protocol and time course for an in vivo model of myocardial ischemia-reperfusion is set forth. Adult, male, WT mice were anesthetized, intubated with a 20 G plastic intravenous catheter and mechanically ventilated. Core body temperature was monitored with a rectal probe and maintained at 37±0.2° C., and a three-lead electrocardiogram was monitored throughout the surgery using a PowerLab data acquisition system (ADInstruments). Mice were positioned in a right lateral decubital position on a heating pad. Using a dissecting microscope, a left thoracotomy was performed on the 4^{th} intercostal space, and the pericardium opened. The LAD coronary artery was reversibly ligated near the origin with a suture (8-0 Monosof MV-135-4, ⅜ 5-mm tapered needle) and subsequently reperfused by release of the ligature. Occlusion was confirmed by sustained S-T wave elevation monitored using a lead-II ECG configuration, regional cyanosis and wall motion abnormalities and reperfusion was confirmed by return of color to the heart distal to the ligation. Two separate sets of experiments were performed. In the first set of experiments, the LAD was ligated for 40 min followed by 2 hours of reperfusion. In the second set of experiments the LAD was ligated for 45 minutes followed by 24 hours of reperfusion.

Five minutes before reperfusion, mice were treated with a single bolus injection of E-WE thrombin (25 μg/kg; iv) or vehicle (PBS) via either the left jugular vein or the femoral vein. For jugular vein access, a PE-10 catheter was directly inserted into the left jugular vein for intravenous drug infusion. For intravenous drug infusion via the femoral vein, a 0.5 mL syringe with a 30 G needle was used to access the vein. This experimental design and treatment paradigm mimics the use of E-WE thrombin administration as a reperfusion therapy.

At the end of either 2 or 24 hours of reperfusion, the LAD was re-occluded and fluorescent polymer microspheres (4 mg/mL in deionized water with 0.01% Tween, diameter 2 to 8 μm) were infused at a rate of 400 μL/min via needle puncture of the left ventricular apex to determine the area at risk. Following fluorescent microsphere perfusion, the heart was excised and cut into 1 mm thick transverse slices, and photographed under UV light to identify the risk area. The infarcted region was identified in the same slices by staining with 2,3,5-triphenyltetrazolium chloride solution (TTC, 1% w/v in sodium phosphate buffer at 37° C., pH 7.4) for 10 minutes and fixing in 10% neutral buffered formalin overnight to optimize the contrast between stained and unstained tissue. Myocardium that did not stain red was considered to be infarcted. Tissue sections were photographed under brightfield and images analyzed in a blinded fashion to calculate the area of myocardium at risk and the infarcted region as a percentage of the left ventricle. Infarct size was normalized as a percentage of the area at risk.

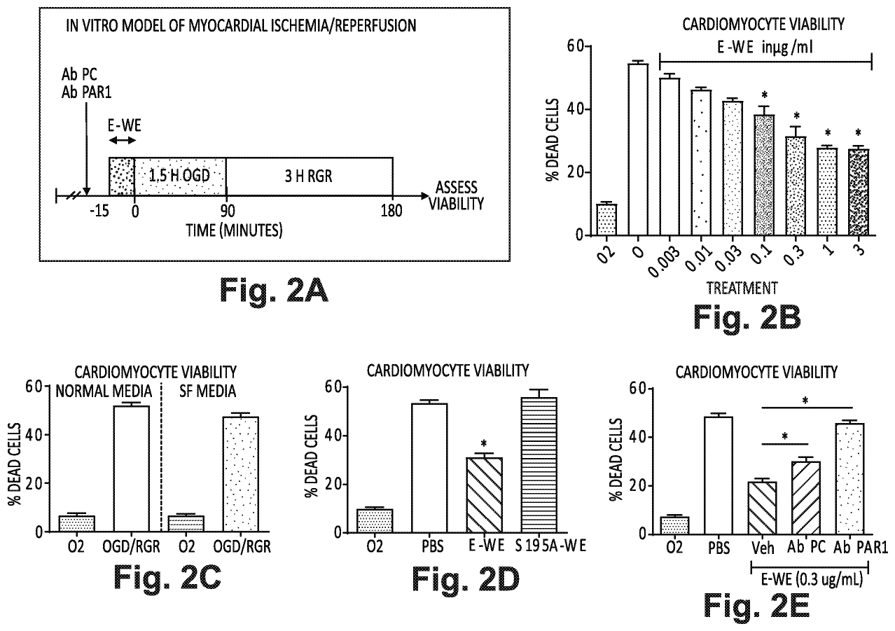

The ischemic area at risk and infarct area were quantified, expressed as the percent of total heart volume (FIG. 1B) and the percentage of area at risk (FIG. 1C), where data are the means±SEM of n=10 mice/group; ***p<0.001. While no difference in area at risk was observed, a single bolus treatment with E-WE thrombin prior to reperfusion significantly reduced infarct size by 37% compared to vehicle control at both 2 and 24 h post-reperfusion. These in vivo studies were complemented by compelling ex vivo data (FIGS. 2B-E) demonstrating that E-WE thrombin confers cardioprotective effects in a simulated ischemia and reperfusion model of oxygen-glucose deprivation/re-oxygenation and glucose repletion (OGD/RGR). Thus, E-WE thrombin reduced myocardial infarct size following experimental ischemia-reperfusion.

E-WE thrombin also improves cardiomyocyte survival following experimental ischemia-reperfusion. In this study, ventricular cardiomyocytes from adult, male, WT mice were prepared and plated based on published methods (O'Connell, T. D., Rodrigo, M. C., Simpson, P. C. (2007) Isolation and culture of adult mouse cardiac myocytes. Methods Mol Biol 357:271-296). The ventricles from three to five hearts were pooled, and cardiomyocytes isolated and cultured as follows. The heart was rapidly excised and the aorta cannulated and perfused with 2 mL Krebs-Henseleit buffer containing 1.2 mM calcium to flush out the remaining blood. Next, the heart was mounted on a heated perfusion apparatus and perfused with a calcium-free buffer containing 2,3-butanedione monoxime (10 mM) to arrest contraction, followed by perfusion with a collagenase 2 solution for 25 minutes to digest the extracellular matrix of the heart. Perfusion with collagenase type 2 was halted after 25 min and the heart removed and submerged into stopping buffer containing 1% bovine serum albumin in calcium-free Krebs-Henseleit perfusion buffer. The hearts were then minced and cells gently dispersed to complete cell isolation. Isolated cardiomyocytes were collected by centrifugation and the fibroblast containing supernatant was discarded. Calcium was re-introduced to a final concentration of 1.2 mM in a three step process. Finally, cardiomyocytes were plated at a density of 30,000 cells/mL in a laminin-coated 24 well plate for viability experiments.

A modified model of oxygen glucose deprivation/reoxygenation glucose repletion (OGD/RGR) was used. To simulate ischemia, glucose-free medium (MEM-HBSS) was pre-equilibrated in 100% N₂ at 37° C. for 2 h. Oxygenated medium was removed from the cardiomyocyte cultures, and replaced with N₂-pre-equilibrated glucose-free medium. Cultures were placed in a Plexiglass™ hypoxia chamber and exposed to 100% N₂ for 1.5 h at 37° C. To simulate reperfusion, glucose-free medium was replaced with M199/10% FBS, and the cells re-oxygenated in 21% O₂/5% CO₂/74% room air for 3 h at 37° C. In a subset of experiments, Opt-mem™ reduced serum media (Thermo Fisher Scientific) was used in place of M199/10% FBS. Cell death was quantified via trypan blue (0.04%) staining, and corrected to number of dead cells in the oxygenated control for each experiment.

To evaluate the cytoprotective potential of E-WE thrombin, cardiomyocytes were pretreated with E-WE thrombin (0.003 to 3 μg/mL) for 15 min prior to OGD/RGR and compared to vehicle control (FIG. 2A). FIG. 2B shows pretreatment with E-WE thrombin prior to OGD improved cell survival in a concentration-dependent manner. Additional experiments (FIGS. 2C-D) confirmed that the protocol was adaptable to serum-free (SF) media with comparable outcomes on cell viability. In the adapted serum-free protocol, E-WE thrombin (0.3 μg/mL) conferred cardioprotective effects, which were dependent on the enzymatic activity of E-WE thrombin since a catalytically inactive analog (S195A E-WE thrombin) had no protective effect.

To evaluate the extent to which E-WE thrombin mediated cardioprotection was dependent on protein C and PAR1 receptor activation, the experiment was repeated in the presence of blocking antibodies. Cells were pretreated with a rat anti-mouse PAR1 antibody (Santa Cruz; 20 μg/mL) or a rat anti-mouse Protein C antibody (10 μg/mL) 5 min prior to treatment with E-WE, thrombin (0.3 μg/mL) and subsequently subjected to the OGD/RGR protocol. Inhibiting PAR1 abolished the cardioprotective effect of E-WE thrombin and inhibiting protein C partially reversed the effect (FIG. 2E). These data confirm the roles of protein C and PAR1 and represent the means±SEM of n=3 experiments: *p<0.05.

Taken together, E-WE thrombin improved cardiomyocyte survival following experimental ischemia/reperfusion. Pretreatment with E-WE thrombin prior to OGD improved cell survival in a concentration-dependent manner. EWE thrombin mediated cardioprotection was protein C and PAR1 dependent since pretreatment with blocking antibodies ameliorated the effect.

### EXAMPLE 2

### E-WE Thrombin Promotes Thrombolysis in a Baboon Thrombosis Model

A well-established baboon model of experimental acute arterial thrombosis was used to determine the relative efficacy of E-WE thrombin to interrupt progressive arterial thrombosis compared with tPA monotherapy. Specifically, this study examined whether treatment with an IV bolus of E-WE thrombin 30 min after thrombus initiation reduced platelet and fibrin deposition (thrombus growth) in a 4 mm i.d. collagen-coated graft that was temporarily deployed into a chronic arteriovenous shunt.

To initiate acute local thrombosis within the AV shunt in baboons, a prosthetic graft segment was interposed within the shunt (Kelly, A. B., et al. (1991) Hirudin interruption of heparin-resistant arterial thrombus formation in baboons. Blood 77(5):1006-12; Schaffer, L. W., et al. (1993) Recombinant leech antiplatelet protein prevents collagen-mediated platelet aggregation but not collagen graft thrombosis in baboons. Arterioscler Thromb 13(11):1593-601). Since local platelet activation inside the lumen of blood vessels can accelerate blood coagulation and initiate thrombosis, the prosthetic vascular grafts were modified to create a surface that predictably and consistently initiates a thrombogenic process, primarily through platelet activation. Since vascular injury exposes flowing blood to the extracellular matrix, which contains structural proteins such as collagen that trigger platelet activation, graft segments were coated with immobilized collagen. Shunts were prepared as follows: the lumens of 20 mm long clinical vascular grafts (expanded-polytetrafluoroethylene, ePTFE, Gore-Tex; W. L. Gore and Associates, Flagstaff, Ariz.) with internal diameters of 4 mm were coated with equine type I collagen (Chronolog Corporation, Haverton, Pa.) for 15 min, and then dried overnight under sterile airflow). This method produces an even collagen coating within the graft. The collagen coated (thrombogenic) graft segments were incorporated into silicon rubber tubing, and deployed into the AV shunts of the baboons).

Blood flow through the shunt in non-anticoagulated baboons consistently triggered acute thrombus formation in the collagen-coated graft segments. During each experiment, maximum blood flow rate through the grafts (typically about 250 mL/min) was restricted by distal clamping to 100 mL/min, producing average initial wall shear rates of 265 s⁻¹ in 4 mm grafts. Flow rate was continuously monitored using an ultrasonic flow meter (Transonics Systems, Ithaca, N.Y.). The 4 mm grafts did not occlude and pulsatile flow rates remained at 100 mL/min during thrombus formation. The graft segment (and thrombus) was removed from the shunt at 90 min and the permanent shunt was restored after each experiment. Since thrombus formation was found to extend downstream from the collagen surface over time, platelet accumulation was also measured within a 10 cm long region of the AV shunt immediately distal to the graft.

Thrombus formation was assessed during the 90 min experiment by quantitative gamma camera imaging of radio-labeled platelets in the graft segment, and further assessed by measurement of endpoint radio-labeled fibrin deposition after termination of each experiment. Briefly, for quantification of platelet deposition, autologous baboon platelets were labeled with 1 mCi of ¹¹¹In, Afterwards, these platelets were re-infused into the animal and allowed to circulate for at least 1 h and up to 4 days before studies were performed. Accumulation of platelet-associated radioactivity onto graft walls was determined at 5-min using a GE-400A-61 gamma scintillation camera interfaced with a NuQuest InteCam computer system. Homologous ¹²⁵I-labeled baboon fibrinogen (5 to 25 μg, 4 uCi, >90% clottable) was intravenously injected 10 min before each study. Incorporation of labeled fibrinogen/fibrin into the thrombus was assessed using a gamma counter (Wizard-3, PerkinEirner, Shelton, Conn.) at least 30 days after removal of the graft from the AV shunt to allow ¹¹¹In attached to platelets to decay.

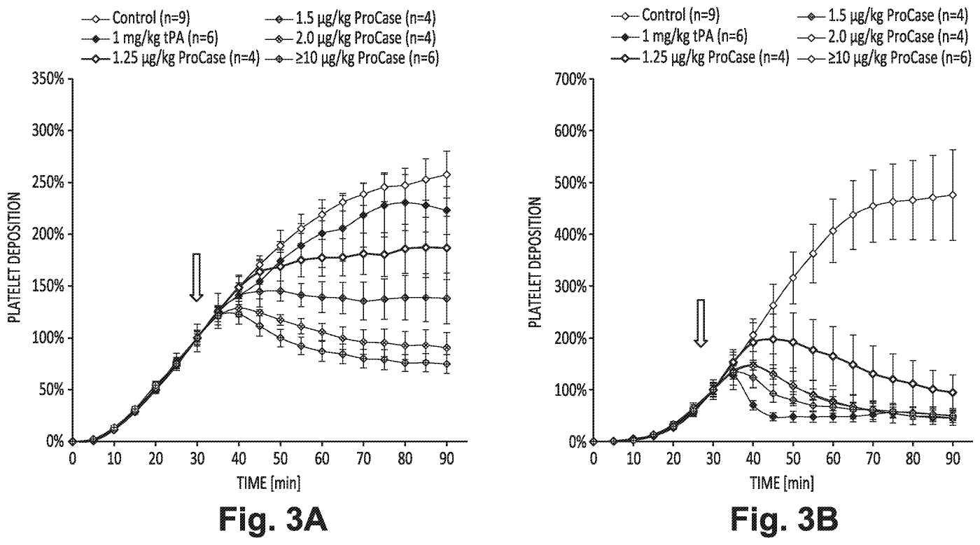

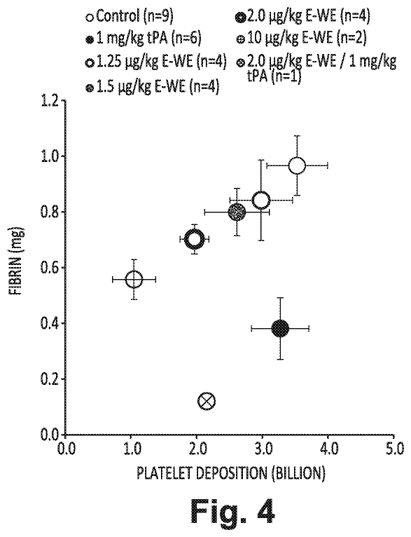

Platelet deposition (FIGS. 3A and 3B) was used to evaluate thrombus growth by measuring platelet deposition in the graft over about a 90 minute period. Radiolabeled platelet deposition in the thrombogenic graft was measured in real-time using gamma camera imaging during experiments. The normalized data with the platelet deposition set to 100% at the time of interruption was evaluated, at which point ˜1.3 billion platelets had already been deposited. The control animals showed steady thrombus growth over the complete 90 min experiment. B-WE thrombin (1.25 to 10 μg/kg) dose-dependently reduced thrombus growth, with the maximum effect observed at ˜2 μg/kg. Bleeding time and volume were also assessed during the studies, with E-WE treated animals showing no increased bleeding compared with controls. However, tPA caused significant ecchymoses that were not observed with E-WE thrombin. Grafts were also analyzed for fibrin content at the end of each study. Compared to the control, E-WE thrombin reduced final platelet deposition and fibrin content of the thrombus in a dose-dependent manner (FIG. 4). Consistent with its mode of action, tPA significantly reduced fibrin content while E-WE thrombin dose-dependently reduced fibrin content in collagen-coated grafts, and enhanced tPA mediated fibrinolysis when compared to the control. E-WE thrombin reduced final platelet deposition and fibrin content of the thrombus in a dose-dependent manner, By contrast, tPA, alone had little effect on platelet deposition and consistent with its mode of action, tPA significantly reduced fibrin content.

### EXAMPLE 3

### E-WE Thrombin/tPA Combination Treatment Enhances Fibrinolvsis in a Baboon Thrombosis Model

The ability of E-WE thrombin to interrupt arterial-type experimental thrombus formation in baboons when combined with a standard interventional dose of tPA (1 mg/kg) was tested. Thrombosis was initiated in the baboons, as described herein, by interposing 4 mm internal diameter collagen coated ePTFE vascular grafts within an arteriolvenous shunt. Thrombus formation was monitored by real-time gamma camera imaging of autologous ¹¹¹In-labelled platelet accumulation in the grafts for a total of 90 min, Fibrin deposition was determined by direct endpoint measurement of incorporated ¹²⁵I-labelled fibrinogen. Antithrombotic interventions were injected intravenously at 30 min after graft deployment into the shunt. Treatment with tPA (1 mg/kg, iv) reduced fibrin deposition by 57%, but did not significantly reduce graft-associated platelet accumulation compared with controls. E-WE thrombin, at doses sequentially ranging from 2 to 10 μg/kg, interrupted thrombus growth within 10 min of treatment, and reduced both platelet and fibrin deposition at 90 min compared with controls in both graft head (FIG. 3A) and tail (FIG. 3B) sections. Thrombus growth was evaluated by measuring platelet deposition in the graft over the course of the 90 minute experiment. E-WE thrombin, tPA, or placebo were administered 30 min after start of experiment as indicated by the arrows in FIGS. 3A and 3B. Compared to control, E-WE thrombin reduced platelet deposition in both graft and tail sections. By contrast, tPA had little effect on platelet deposition in the head section, but significantly reduced platelet deposition in the tail. E-WE thrombin dose-dependently reduced thrombus growth in collagen-coated grafts.

As shown, when E-WE thrombin (2 μg/kg) was co-administered with tPA (1 mg/kg), the result observed was a profound 91% reduction in thrombus fibrin content, with platelet deposition being reduced by 34% compared with controls (FIG. 4).

The effect of the test article on the primary hemostasis of baboons was assessed using the standard template bleeding time test (Surgicutt, International Technidyne Corp.) following manufacturer's instruction. Bleeding time was recorded by the technician performing the test using a manual stop watch. Blood drops emerging from the wound were collected every 30 sec using a Whatman™ blotting paper. Bleeding volume was assessed using these blotting papers and Drabkin's reagent (SiomaAldrich), a quantitative, colorimetric chemical that allows for determination of hemoglobin concentration in whole blood. The dried blood sample on the blotting paper was soaked in 2.5 mL Drabkin's reagent until completely dissolved. Absorbance was measured at 540 nm and compared to a standard curve using blood of the tested animal. E-WE thrombin treated animals showed no increased bleeding compared with controls. The combination therapy showed no overt anti-hemostatic effects beyond tPA administration alone. These data support that E-WE thrombin inhibits TAFI activation, and co-administration of E-WE thrombin with tPA improves the efficacy of thrombolysis without additional hemostasis impairment.

### EXAMPLE 4

### E-WE Thrombin Competitively Inhibits TAFI Activation by Thrombin In Vitro

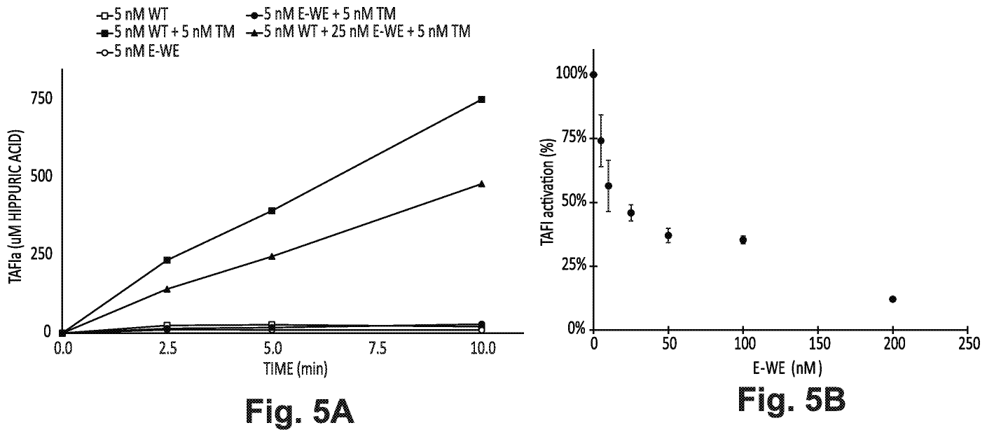

Activated TAFI produced over time by WT thrombin and E-WE thrombin in the presence or absence of TM was measured by quantification of hippuric acid (FIG. 5A). Quantitative activation of TAFI was measured as previously described by Mosnier (Mosnier et al. (2011) J. Biol. Chem. 286:502-510). A five-fold molar excess of E-WE thrombin was added to WT thrombin to assess inhibition of TAFI activation by E-WE thrombin over the incubation period. E-WE thrombin was added to 5 nM WT thrombin and 5 nM TM in increasing doses from 5 to 200 nM for 10 min and active TAFI measured by quantification of hippuric acid (FIG. 5B). Activated TAFI produced by WT thrombin in the absence of E-WE thrombin was set to 100% and the relative activity for increasing does of E-WE thrombin measured against this reference.

Briefly, purified TAFI (300 nM) was incubated with WT α-thrombin (5 nM) and/or E-WE thrombin (5 nM to 200 nM, depending on experiment) with or without thrombomodulin (5 nM) in 60 μL HEPES-buffered saline (20 mM HEPES, 150 mM NaCl, 5 mM CaCl₂, pH 7.4) at room temperature for 2.5 to 10 min after which 20 μL of 150 μM PPACK was added. Samples were then incubated with 20 _{μL} of 20 mM hippuryl-arginine for 10 min at room temperature, reactions quenched with 20 μL of 1 M HCl, neutralized with 20 μL of 1 M NaOH, and supplemented with 25 μL of 2 M sodium phosphate (pH 7.4) to facilitate colorimetric determination of hippuric acid with cyanuric chloride. To measure this conversion, 60 μL of 3% cyanuric chloride in dioxane were added to each sample and hippuric acid standards and incubated with vortexing until color developed. The absorbance of centrifugation-cleared samples was measured at 382 nm and the amount of hippuric acid generated was calculated as a proxy for activated TAFI. These experiments demonstrated that E-WE thrombin is a poor activator of TAFI and that it competitively inhibits thrombin/thrombomodulin-mediated activation of TAFI.

### EXAMPLE 5

### E-WE Thrombin Accelerates Clot Lysis Induced by Tissue Plasminogen Activator (tPA) in Normal Baboon Plasma

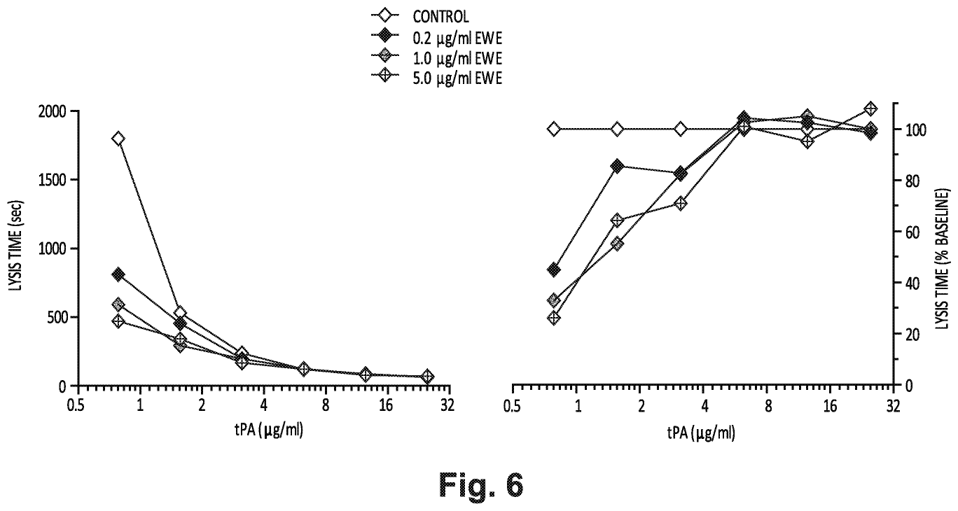

Clot lysis time was measured to evaluate the effects of E-WE thrombin on the fibrinolytic activity of tPA-spike plasma in vitro. Pooled baboon plasma from 3 naïve baboons was spiked with varying concentrations of t-PA (0.78 to 25 μg/mL) combined with various concentrations of E-WE thrombin (0 to 5 μg/mL) in a total volume of 0.1 mL. The clot formed during aPTT measurement was monitored post clot to assess the time until clot lysis, i.e., the plasma clot completely liquefies. The clot was formed using standard, commercially available aPTT reagents (SynthASil, Instrumentation Laboratory, Bedford, Mass.) and platelet poor plasma spiked with tPA alone or tPA plus E-WE thrombin. Clot lysis time was reduced with increasing concentrations of tPA, from 1800 sec at 0.78 μg/mL to less than 60 sec at 25 μg/mL of tPA added to the plasma (FIG. 6). E-WE thrombin accelerated the lysis time by more than 2-fold (>50% reduction) at the lowest tPA concentration

The above-disclosed protocols and procedures in each of the examples set forth herein were performed with E-WE thrombin composing the amino acid sequence set forth in SEQ ID NO:1 Performance with alternative thrombin analogs as disclosed herein, e.g., E-WE thrombin comprising the amino acid sequence set forth in SEQ ID NO:22 and WE thrombin comprising the amino acid sequence set forth m SEQ ID NO:2, is reasonably expected to provide similar results. In use, when administered to a subject experiencing an acute thrombotic episode, WE or E-WE thrombin in combination with fibrinolytics as disclosed herein can save lives and reduce the long-term effects of, e.g., stroke, heart attack, pulmonary embolism, and other acute thrombotic emergencies. WE or E-WE thrombin combined with plasminogen activators may be administered, e.g., combined either into a single compound or formulation, or administered sequentially, and may be administered, for example, systemically or locally through an IV, to inhibit thrombus formation and/or dissolve a thrombus and ii prove blood flow to the region of the subject being deprived of blood.

LISTED SEQUENCES

E-WE thrombin
SEG ID NO.: 1
TFGSGEADCG LRPLFEKKSL EDKTERELLE SYIDGRIVEG
SDAEIGMSPW QVMLFRKSPQ ELLCGASLIS DRWVLTAAHC
LLYPPWDKNF TENDLLVRIG KHSRTRYERN IEKISMLEKI
YIHPRYNWRE NLDRDIALMK LKKPVAFSDY IHPVCLPDRE
TAASLLQAGY KGRVTGWGNL KETWTANVGK GQPSVLGVVN
LPIVERPVCK DSTRIRITDN MFCAGYKPDE GKRGDACEGD
SGGPFVMKSP FNNRWYQMGI VSAGAGCDRD GKYGFYTHVF
RLKKWIQKVI DQFGE
WE Thrombin
SEQ ID NO: 2
TFGSGEADCG LRPLFEKKSL EDKTERELLE SYIDGRIVEG
SDAEIGMSPW QVMLFRKSFQ ELLCGASLIS DRWVLTAAHC
LLYPPWDKNF TENDLLVRIG KHSRTRYERN IEKISMLEKI
YIHFRYNWRE NLDRDIALMK LKKPVAFSDY IHPVCLPDRE
TAASLLQAGY KGRVTGWGNL KETWTANVGK GQPSVLQVVN
LPIVERPVCK DSTRIRITDN MFCAGYKPDE GKRGDACEGD
SGGPFVMKSP FNNRVVMMGI VSAGAGCDRO GKYGFYTHVF
RLKKWIQKVI DQFGE
Thrombin
SEQ ID NO: 3
TFGSGEADCG LRPLFEKKSL EDKTERELLE SYIDGRIVEG
SDAEIGMSPW QVMLFRKSPQ ELLCGASLIS DRWVLTAAHC
LLYPPWDKNF TENDLLVRIG KHSRTRYERN IEKISMLEKI
YIHPRYNWRE NLDRDIALMK LKKPVAFSDY IHPVCLPDRE
TAASLLQAGY KGRVTGWGNL KETWTANVGK GQRSVLQVVN
LPIVERPVCK DSTRIRITDN MFCAGYKPDE GKRGDACEGD
SGGPFVMKSP FNNRMQMGI VSWGEGCDRD GKYGFYTHVF
RLKKWIQKVI DQFGE
Preprothrombin
SEQ ID NO: 4
MAHVRGLQLP GCLALAALCS LVHSQHVFLA PQQARSLLQR
VRRANTFLEE VRKGNLEREC VEETCSYEEA FEALESSTAT
DVFWAKYTAC ETARTPRDKL AACLEGNCAE GLGTNYRGHV
NITRSGIECQ LWRSRYPHKP EINSTTHPGA DLQENFCRNP
DSSTTGPWCY TTDPTVRRQE CSIPVCGQDQ VTVAMTPRSE
GSSVNLSPPL EQCVPDRGQQ YQGRLAVTTH GLPCLAWASA
QAKALSKHQD FNSAVQLVEN FCRNPDGDEE GVWCYVAGKP
GDFGYCDLNY CEEAVEEETG DGLDEDSDRA IEGRTATSEY
QTFFNPRTFG SGEADCGLRP LFEKKSLEDK TERELLESEY
DGRIVEGSDA EIGMSPWQVM LFRKSPQELL CGASLISDRW
VLTAAHCLLY PPWDKNFTEN DLLVRIGKHS RTRYERNIEK
ISMLEKIYIH PRYNWRENLD RDIALMKLKK PVAFSDYIHP
VCLPDRETAA SLLQAGYKGR VTGWGNLKET WTANVGKGQP
SVLQVVNLPI VERPVCKDST RIRITDNMFC AGYKPDEGKR
GDACEGDSGG PFVMKSPFNN RWYQMGIVSW GEGCDRDGKY
GFYTHVFRLK KWIQKVIDQF GE
Ecarin-activatable E-WE Preprothrombin
SEQ ID NO: 5
MAHVRGLQLP GCLALAALCS LVHSQHVFLA PQQARSLLQR
VRRANTFLEE VRKGNLEREC VEETCSYEEA FEALESSTAT
DVFWAKYTAC ETARTPRDKL AACLEGNCAE GLGTNYRGHV
NITRSGIECQ LWRSRYPHKP EINSTTHPGA DLQENFCRNP
DSSTTGPWCY TTDPTVRRQE CSIPVCGQDQ VTVAMTPRSE
GSSVNLSPPL EQCVPDRGQQ YQGRLAVTTH GLPCLAWASA
QAKALSKHQD FNSAVQLVEN FCRNPDGDEE GVWCYVAGKP
GDFGYCDLNY CEEAVEEETG DGLDEDSDRA IEGRTATSEY
QTFFDGRTFG SGEADCGLRP LFEKKSLEDK TERELLESYI
DGRIVEGSDA EIGMSPWQVM LFRKSPQELL CGASLISDRW
VLTAAHCLLY PPWDKNFTEN DLLVRIGKHS RTRYERNIEK
ISMLEKIYIH PRYNWRENLD RDIALMKLKK PVAFSDYIHP
VCLPDRETAA SLLQAGYKGR VTGWGNLKET WTANVGKGQP
SVLQVVNLPI VERPVCKDST RIRITDNMFC AGYKPDEGKR
GDACEGDSGG PFVMKSPFNN RWYQMGIVSA GAGCDRDGKY
GFYTHVFRLK KWIQKVIDQF GE
Ecarin-activatable EWE Prethrombin-2
SEQ ID NO: 6
TATSEYQTFF DGRTFGSGEA DCGLRPLFEK KSLEDKTERE
LLESYIDGRI VEGSDAEIGM SPWQVMLFRK SPQELLCGAS
LISDRWVLTA AHCLLYPPWD KNFTENDLLV RIGKHSRTRY
ERNIEKISML EKIYIHPRYN WRENLDRDIA LMKLKKPVAF
SDYIHPVCLP DRETAASLLQ AGYKGRVTGW GNLKETWTAN
VGKGQPSVLQ VVNLPIVERP VCKDSTRIRI TDNMFCAGYK
PDEGKRGDAC EGDSGGPFVM KSPFNNRWYQ MGIVSAGAGC
DRDGKYGFYT HVFRLKKWIQ KVIDQFGE
Ecarin-activatable Preprothrombin
SEQ ID NO: 7
MAHVRGLQLP GCLALAALCS LVHSQHVFLA PQQARSLLQR
VRRANTFLEE VRKGNLEREC VEETCSYEEA FEALESSTAT
DVFWAKYTAC ETARTPRDKL AACLEGNCAE GLGTNYRGHV
NITRSGIECQ LWRSRYPHKP EINSTTHPGA DLQENFCRNP
DSSTTGPWCY TTDPTVRRQE CSIPVCGODQ VTVAMTPRSE
GSSVNLSPPL EQCVPDRGQQ YQGRLAVTTH GLPCLAWASA
QAKALSKHQD FNSAVQLVEN FCRNPDGDEE GVWCYVAGKP
GDFGYCDLNY CEEAVEEETG DGLDEDSDRA IEGRTATSEY
QTFFDGRTFG SGEADCGLRP LFEKKSLEDK TERELLESYI
DGRIVEGSDA EIGMSPWQVM LFRKSPQELL CGASL1SDRW
VLTAAHCLLY PPWDKNFTEN DLLVRIGKHS RTRYERN1EK
ISMLEKIYIH PRYNWRENLD RDIALMKLKK PVAFSDYIHP
VCLPDRETAA SLLQAGYKGR VTGWGNLKET WTANVGKGQP
SVLQVVNLPI VERPVCKDST RIRITDNMFC AGYKPDEGKR
GDACEGDSGG PFVMKSPFNN RVWQMGIVSW GEGCDRDGKY
GFYTHVFRLK KWIQKVIDQF GE
Ecarin-activatable Prethrombin-2
SEQ ID NO: 8
TATSEYQTFF DGRTFGSGEA DCGLRPLFEK KSLEDKTERE
LLESYIDGRI VEGSDAEIGM SPWQVMLFRK SPQELLCGAS
LISDRWVLTA AHCLLYPPWD KNFTENDLLV RIGKHSRTRY
ERNIEKISML EKIYIHPRYN WRENLDRDIA LMKLKKPVAF
SDYIHPVCLP DRETAASLLQ AGYKGRVTGW GNLKETWTAN
VGKGQPSVLQ VVNLPIVERP VCKDSTRIRI TDNMFCAGYK
PDEGKRGDAC EGDSGGPFVM KSPENNRWYQ MGIVSWGEGC
DRDGKYGFYT HVFRLKKWIQ KVIDQFGE
Ecarin-activatable Δ146-149e Prethrombin-2
SEQ ID NO: 9
TATSEYQTFF DGRTFGSGEA DCGLRPLFEK KSLEDKTEPE
LLESYIDGRI VEGSDAEIGM SPWQVMLFRK SPQELLCGAS
LISDRWVLTA AHCLLYPPWD KNFTENDLLV RIGKHSRTRY
ERNIEKISML EKIYIHPRYN WRENLDRDIA LMKLKKPVAF
SDYIHPVCLP DRETAASLLQ AGYKGRVTGW GNLKGKGQPS
VLQVVNLPIV ERPVCKDSTR IRITDNMFCA GYKPDEGKRG
DACEGDSGGP FVMKSPFNNR WYQMGIVSWG EGCDRDGKYG
FYTHVFRLKK WIQKVIDQFG E
WE Preprothrombin
SEQ ID NO: 10
MAHVRGLQLP GCLALAALCS LVHSQHVFLA PQQARSLLQR
VRRANTFLEE VRKGNLEREC VEETCSYEEA FEALESSTAT
DVFWAKYTAC ETARTPRDKL AACLEGNCAE GLGTNYRGHV
NITRSGIECQ LWRSRYPHKP EINSTTHPGA DLOENFCRNP
DSSTTGPWCY TTDPTVRRQE CSIPVCGQDQ VTVAMTPRSE
GSSVNLSPPL EQCVPDRGQQ YQGRLAVTTH GLPCLAWASA
QAKALSKHQD FNSAVQLVEN FCRNPDGDEE GVWCYVAGKP
GDFGYCDLNY CEEAVEEETG DGLDEDSDRA IEGRTATSEY
QTFFNPRTFG SGEADCGLRP LFEKKSLEDK TERELLESYI
DGRIVEGSDA EIGMSPWQVM LFRKSPQELL CGASLISDRW
VLTAAHCLLY PPWDKNFTEN DLLVR1GKHS RTRYERNIEK
ISMLEKIYIH PRYNWRENLD RDIALMKLKK PVAFSDYIHP
VCLPDRETAA SLLQAGYKGR VTGWGNLKET WTANVGKGQP
SVLQVVNLP1 VERPVCKDST RIRITDNMFC AGYKPDEGKR
GDACEGDSGG PFVMKSPFNN RWYQMGIVSA GAGCDRDGKY
GFYTHVFRLK KWIQKVIDQF GE
WE Prethrombin-2
SEQ ID NO: 11
TATSEYQTFF NPRTFGSGEA DCGLRPLFEK KSLEDKTERE
LLESYIDGRI VEGSDAEIGM SPWQVMLFRK SPQELLCGAS
LISDRWVLTA AHCLLYPPWD KNFTENDLLV RIGKHSRTRY
ERNIEKISML EKIYIHPRYN WRENLDRDIA LMKLKKPVAF
SDYIHPVCLP DRETAASLLQ AGYKGRVTGW GNLKETWTAN
VGKGQPSVLQ VVNLPIVERP VCKDSTRIRI TDNMFCAGYK
PDEGKRGDAC EGDSGGPFVM KSPFNNRWYQ MGIVSAGAGC
DRDGKYGFYT HVFRLKKWIQ KVIDQFGE
SEQ ID NO: 12
FNPRTF
SEQ ID NO: 13
FDGRTF
SEQ ID NO: 14
YIDGRIV
Ecarin-activatable E-WE Thrombin Precursor A
SEQ ID NO: 15
DGRTFGSGEA DCGLRPLFEK KSLEDKTERE LLESYIDGRI
VEGSDAEIGM SPWQVMLFRK SPQELLCGAS LISDRWVLTA
AHCLLYPPWD KNFTENDLLV RIGKHSRTRY ERN1EKISML
EKIYIHPRYN WRENLDRDIA LMKLKKPVAF SDYIHPVCLP
DRETAASLLQ AGYKGRVTGW GNLKETWTAN VGKGQPSVLQ
VVNLPIVERP VCKDSTRIRI TDNMFCAGYK PDEGKRGDAC
EGDSGGPFVM KSPFNNRWYQ MGIVSAGAGC DRDGKYGFYT
HVFRLKKWIQ KVIDQFGE
SEQ ID NO: 16
EGRTFGSGEA DCGLRPLFEK KSLEDKTERE LLESYIDGRI
VEGSDAEIGM SPWQVMLFRK SPQELLCGAS LISDRWVLTA
AHCLLYPPWD KNFTENDLLV RIGKHSRTRY ERNIEKISML
EKIYIHPRYN WRENLDRD1A LMKLKKPVAF SDYIHPVCLP
DRETAASLLQ AGYKGRVTGW GNLKETWTAN VGKGQPSVLQ
VVNLPIVERP VCKDSTRIRI TDNMFCAGYK PDEGKRGDAC
EGDSGGPFVM KSPFNNRWYQ MG1VSAGAGC DRDGKYGFYT
HVFRLKKWIQ KVIDQFGE
Ecarin-activatable Thrombin Precursor A
SEQ ID NO: 17
DGRTFGSGEA DCGLRPLFEK KSLEDKTERE LLESYIDGRI
VEGSDAEIGM SPWOVMLFRK SPQELLCGAS LISDRWVLTA
AHCLLYPPWD KNFTENDLLV RIGKHSRTRY ERNIEKISML
EKIYIHPRYN WRENLDRDIA LMKLKKPVAF SDYIHPVCLP
DRETAASLLQ AGYKGRVTGW GNLKETWTAN VGKGQPSVLQ
VVNLPIVERP VCKDSTRIRI TDNMFCAGYK PDEGKRGDAC
EGDSGGPFVM KSPFNNRWYQ MGIVSWGEGC DRDGKYGFYT
HVFRLKKWIQ KVIDQFGE
SEQ ID NO: 18
EGRTFGSGEA DCGLRPLFEK KSLEDKTERE LLESYIDGRI
VEGSDAEIGM SPWQVMLFRK SPQELLCGAS LISDRWVLTA
AHCLLYPPWD KNFTENDLLV RIGKHSRTRY ERNIEKISML
EKIYIHPRYN WRENLDRDIA LMKLKKPVAF SDYIHPVCLP
DRETAASLLQ AGYKGRVTGW GNLKETWTAN VGKGQPSVLQ
VVNLPIVERP VCKDSTRIRI TDNMFCAGYK PDEGKRGDAC
EGDSGGPFVM KSPFNNRWY0 MGIVSWGEGC DRDGKYGFYT
HVERLKKWIQ KVIDQFGE
WE Prothrombin
SEQ ID NO: 19
ANTFLEEVRK GNLERECVEE TCSYEEAFEA LESSTATDVF
WAKYTACETA RTPRDKLAAC LEGNCAEGLG TNYRGHVNIT
RSG1ECQLWR SRYPHKPEIN STTHPGADLQ ENFCRNPDSS
TTGPWCYTTD PTVRRQECSI PVCGQDQVTV AMTPRSEGSS
VNLSPPLEQC VPDRGQQYQG RLAVTTHGLP CLAWASAQAK
ALSKHQDFNS AVQLVENFCR NPDGDEEGVW CYVAGKPGDF
GYCDLNYCEE AVEEETGDGL DEDSDRAIEG RTATSEYQTF
FNPRTFGSGE ADCGLRPLFE KKSLEDKTER ELLESY1DGR
IVEGSDAEIG MSPWQVMLER KSPQELLCGA SLISDRWVLT
AAHCLLYPPW DKNFTENDLL VRIGKHSRTR YERNIEKISM
LEKIYIHPRY NWRENLDRDI ALMKLKKPVA FSDYIHPVCL
PDRETNASLL QAGYKGRVTG WGNLKETWFA NVGKGQPSVL
QVVNLPIVER PVCKDSTRIR ITDNMFCAGY KPDEGKRGDA
CEGDSGGPFV MKSPFNNRWY QMGIVSAGAG CDRDGKYGFY
THVFRLKKWI QKVIDQFGE
Ecarin-activatable EWE Prothrombin
SEQ ID NO: 20
ANTFLEEVRK GNLERECVEE TCSYEEAFEA LESSTATDVF
WAKYTACETA RTPRDKLAAC LEGNCAEGLG TNYRGHVNIT
RSGIECQLWR SRYPHKPEIN STTHPGADLQ ENFCRNPDSS
TTGPWCYTTD PTVRRQECSI PVCGODQVTV AMTPRSEGSS
VNLSPPLEQC VPDRGQQYQG RLAVTTHGLP CLAWASAOAK
ALSKHQDFNS AVQLVENFCR NPDGDEEGVW CYVAGKPGDF
GYCDLNYCEE AVEEETGDGL DEDSDRAIEG RTATSEYOTF
FDGRTFGSGE ADCGLRPLFE KKSLEDKTER ELLESYIDGR
IVEGSDAEIG MSPWQMILFR KSPQELLCGA SUSDRVVVLT
AAHCLLYPPW DKNFTENDLL VRIGKHSRTR YERNIEKISM
LEKIYIHPRY NWRENLDRDI ALMKLKKPVA FSDY1HPVCL
PDRETAASLL QAGYKGRVTG WGNLKETWTA NVGKGQPSVL
CANNLPIVER PVCKDSTRIR ITDNMFCAGY KPDEGKRGDA
CEGDSGGPFV MKSPFNNRWY QMGIVSAGAG CDRDGKYGFY
THVFRLKKWI QKVIDQFGE
SEQ ID NO: 21
ANTFLEEVRK GNLERECVEE TCSYEEAFEA LESSTATDVF
WAKYTACETA RTPRDKLAAC LEGNCAEGLG TNYRGHVNIT
RSGIECQLWR SRYPHKPEIN STTHPGADLQ ENFCRNPDSS
TTGPWCYTTD FTVRRQECSI PVCGQDQVTV AMTPRSEGSS
VNLSPPLEQC VPDRGQQYQG RLAVTTHGLP CLAWASAQAK
ALSKHQDFNS AVQLVENFCR NPDGDEEGVW CYVAGKPGDF
GYCDLNYCEE AVEEETGDGL DEDSDRA1EG RTATSEYQTF
FEGRTFGSGE ADCGLRPLFE KKSLEDKTER ELLESYIDGR
IVEGSDAEIG MSPWQVMLFR KSPQELLCGA SLISDRWVLT
AAHCLLYPPW DKNFTENDLL VRIGKHSRTR YERNIEKISM
LEKIYIHPRY NWRENLDRDI ALMKLKKPVA FSDYIHPVCL
PDRETAASLL QAGYKGRVTG WGNLKETWFA NVGKGQPSVL
QVVNLPIVER PVCKDSTRIR ITDNMFCAGY KPDEGKRGDA
CEGDSGGPEV MKSPFNNRVW QMGIVSAGAG CDRDGKYGFY
THVFRLKKWI QKVIDQFGE
E-WE Thrombin with Ecarin Site
SEQ ID NO: 22
DGRTFGSGE ADCGLRPLFE KKSLEDKTER ELLESYIDGR
IVEGSDAEIG MSPWQVMLFR KSPQELLCGA SLISDRWVLT
AAHCLLYPPW DKNFTENDLL VRIGKHSRTR YERNIEKISM
LEKIYIHPRY NWRENLDRDI ALMKLKKPVA FSDYIHPVCL
PDRETAASLL QAGYKGRVTG WGNLKETWTA NVGKGQPSVL
QVVNLPIVER PVCKDSTRIR ITDNMFCAGY KPDEGKRGDA
CEGDSGGFEV MKSPENNRWY QMGIVSAGAG CDRDGKYGFY
THVERLKKWI QKVIDQFGE
SEQ ID NO: 23
EGRTFGSGE ADCGLRFLFE KKSLEDKTER ELLESYIDGR
IVEGSDAEIG MSPWQMILFR KSPQELLCGA SLISDRWVLT
AAHCLLYPPW DKNFTENDLL VRIGKHSRTR YERNIEKISM
LEKIYIHPRY NWRENLDRDI ALMKLKKPVA FSDYIHPVCL
PDRETAASLL QAGYKGRVTG WGNLKETWTA NVGKGQPSVL
CANNLFIVER FVCKDSTRIR ITDNMFCAGY KPDEGKRGDA
CEGDSGGFFV MKSPENNRWY QMGIVSAGAG CDRDGKYGFY
THVERLKKWI QKVIDQFGE
SEQ. ID NO: 24
EGRTFGSGE ADCGLRFLFE KKSLEDKTER ELLESYIDGR
IVEGSDAEIG MSPWQVMLFR KSPQELLCGA SLISDRWVLT
AAHCLLYPPW DKNFTENDLL VRIGKHSRTR YERNIEKISM
LEKIYIHPRY NWRENLDRDI ALMKLKKPVA FSDYIHPVCL
PDRETAASLL QAGYKGRVTG WGNLKETWTA NVGKGQPSVL
OVVNLPIVER FVCKDSTRIR ITDNMECAGY KPDEGKRGDA
CEGDSGGPFV MKSPFNNRWY QMGIVSWGEG CDRDGKYGFY
THVERLKKWI QKVIDOFGE

All references, including publications, patent applications, and patents, cited herein are hereby incorporated by reference to the same extent as if each reference were individually and specifically indicated to be incorporated by reference and were set forth in its entirety herein.

A number of variations and modifications of the disclosure can be used. It would be possible to provide for some features of the disclosure without providing others. The present disclosure, in various aspects, embodiments, and configurations, includes components, methods, processes, systems and/or apparatus substantially as depicted and described herein, including various aspects, embodiments, configurations, subcombinations, and subsets thereof. Those of skill in the art understand how to make and use the various aspects, aspects, embodiments, and configurations, after understanding the present disclosure. The present disclosure, in various aspects, embodiments, and configurations, includes providing devices and processes in the absence of items not depicted and/or described herein or in various aspects, embodiments, and configurations hereof, including in the absence of such items as may have been used in previous devices or processes, for example, for improving performance, achieving ease and/or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more, aspects, embodiments, and configurations for the purpose of streamlining the disclosure. The features of the aspects, embodiments, and configurations of the disclosure may be combined in alternate aspects, embodiments, and configurations other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed aspects, embodiments, and configurations. Thus, the following claims are hereby incorporated into this Detailed Description, with each claim standing on its own as a separate preferred embodiment of the disclosure.

Moreover, though the description of the disclosure has included description of one or more aspects, embodiments, or configurations and certain variations and modifications, other variations, combinations, and modifications are within the scope of the disclosure, for example, as may be within the skill and knowledge of those in the art, after understanding the present disclosure. It is intended to obtain rights which include alternative aspects, embodiments, and configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

We claim:

1. A pharmaceutically acceptable composition for promoting thrombus dissolution in a subject, the composition comprising:
at least one antithrombotic thrombin analog, and
at least one fibrinolytic agent.

2. The composition according to claim 1, wherein the at least one thrombin analog comprises a WE thrombin analog.

3. The composition according to claim 2, wherein the at east one WE thrombin analog comprises the amino acid sequence set forth in SEQ ID NO:2.

4. The composition according to claim 1, wherein the at least one thrombin analog comprises an E-WE thrombin analog.

5. The composition according to claim 4, wherein the at least one E-WE thrombin analog comprises the arnino acid sequence set forth in SEC) ID NO:1.

6. The composition according to claim 4, wherein the at least one E-WE thrombin analog comprises the amino add sequence set forth in SEQ ID NO:22.

7. The composition according to claim 1, wherein the at least one fibrinolytic agent is selected from the group consisting of scuPA, tPA, uPA, tcuPA, streptokinase, rt-PA, alteplase, rt-PA derivatives, reteplase, lanoteplase, TNK-re-PA, anisoylated plasminogen streptokinase complex, anistreplase, streptokinase derivative, and combinations thereof.

8. The composition according to claim 7, wherein the at least one fibrinolytic agent comprises tPA.

9. The composition according to claim 1, wherein the at least one thrombin analog comprises the amino acid sequence set forth in SEQ ID NO:2, and the at least one fibrinolytic agent is selected from the group consisting of scuPA, tPA, uPA, tcuPA, streptokinase, rt-PA, alteplase, rt-PA derivatives, reteplase, lanoteplase, TNK-re-PA, anisoylated plasminogen streptokinase complex, anistreplase, streptokinase derivative, and combinations thereof.

10. The composition according to claim 1, wherein the at least one thrombin analog comprises the amino acid sequence set forth in SEC) ID NO:1, and the at least one fibrinolytic agent is selected from the group consisting of scuPA, tPA, uPA, tcuPA, streptokinase, rt-PA, alteplase, rt-PA derivatives, reteplase, lanoteplase, TNK-re-PA, anisoylated plasminogen streptokinase complex, anistreplase, streptokinase derivative, and combinations thereof.

11. The composition according to claim 1, wherein the at least one thrombin analog comprises the amino acid sequence set forth in SEQ ID NO:22, and the at least one fibrinolytic agent is selected from the group consisting of scuPA, tPA, uPA, tcuPA, streptokinase, rt-PA, alteplase, rt-PA derivatives, reteplase, lanoteplase, TNK-re-PA, anisoylated plasminogen streptokinase complex, anistreplase, streptokinase derivative, and combinations thereof.

12. A pharmaceutically acceptable composition for promoting thrombus dissolution in a subject, the composition comprising:
a thrombin analog, wherein the analog comprises a WE thrombin analog comprising the amino acid sequence as set forth in SEQ ID NO:2: and,
a fibrinolytic agent, wherein the agent comprises tPA.

13. A pharmaceutically acceptable composition for promoting thrombus dissolution in a subject, the composition comprising:
a thrombin analog, wherein the analog comprises an E-WE thrombin analog comprising the amino acid sequence as set forth in SEQ ID NO:1; and,
a fibrinolytic agent, wherein the agent comprises tPA.

14. A pharmaceutically acceptable composition for promoting thrombus dissolution in a subject, the composition comprising:
a thrombin analog, wherein the analog comprises an E-WE thrombin analog comprising the amino acid sequence as set forth in SEQ ID NO:22; and,
a fibrinolytic agent, wherein the agent comprises tPA.

15. A method of enhancing fibrinolysis in a subject having a thrombotic or thromboembolic disorder, the method comprising the steps of administering to the subject a pharmaceutically acceptable composition comprising an effective dosage of at least one antithrombotic thrombin analog prior to, subsequent to, or concurrently with an effective dosage of at least one fibrinolytic agent.

16. The method according to claim 15, wherein the at least one thrombin analog comprises a WE thrombin analog.

17. The method according to claim 16, wherein the at least one WE thrombin analog comprises the amino acid sequence set forth in SEQ ID NO:2.

18. The method according to claim 15, wherein the at least one thrombin analog comprises an E-WE thrombin analog.

19. The method according to claim 18, wherein the at least one E-WE thrombin analog comprises the amino acid sequence set forth in SEQ ID NO:1.

20. The method according to claim 18, wherein the at least one E-WE thrombin analog comprises the amino acid sequence set forth in SEQ ID NO:22.

21. The method of claim 15, wherein the at least one antithrombotic thrombin analog comprises the amino acid sequence set forth in SEQ ID NO:2, and
wherein the at least one fibrinolytic agent is selected from the group consisting of scuPA, tPA, uPA, tcuPA, streptokinase, rt-PA, alteplase, rt-PA derivatives, reteplase, lanoteplase, TNK-re-PA, anisoylated plasminogen streptokinase complex, anistreplase, streptokinase derivative, and combinations thereof.

22. The method of claim 15, wherein the at least one antithrombotic thrombin analog comprises the amino acid sequence set forth in SEQ ID NO:1, and wherein the at least one fibrinolytic agent is selected from the group consisting of scuPA, tPA, uPA, tcuPA, streptokinase, rt-PA, alteplase, derivatives, reteplase, lanoteplase, TNK-re-PA, anisoylated plasminogen streptokinase complex, anistreplase, streptokinase derivative, and combinations thereof.

23. The method of claim 15, wherein the at least one antithrombotic thrombin analog comprises the amino acid sequence set forth in SEQ ID NO:22, and
wherein the at least one fibrinolytic agent is selected from the group consisting of scuPA, tPA, uPA, tcuPA, streptokinase, rt-PA, alteplase, rt-PA derivatives, reteplase, lanoteplase, TNK-re-PA, anisoylated plasminogen streptokinase complex, anistreplase, streptokinase derivative, and combinations thereof.

24. The method according to claim 15, where in the at least one antithrombotic thrombin analog is comprised of at least one WE thrombin analog comprising the amino acid sequence set forth in SEQ ID NO:2 and the at least one fibrinolytic agent comprises tPA.

25. The method according to claim 15, where in the at least one antithrombotic thrombin analog is comprised of at least one E-WE thrombin analog comprising the amino acid sequence set forth in SEQ ID NO:1 and the at least one fibrinolytic agent comprises tPA.

26. The method according to claim 15, where in the at least one antithrombotic thrombin analog is comprised of at least one E-WE thrombin analog comprising the amino acid sequence set forth in SEQ ID NO:22 and the at least one fibrinolytic agent comprises tPA.

27. A kit comprising:
a pharmaceutically acceptable composition for promoting thrombus dissolution in a subject, the composition comprising at least one antithrombotic thrombin analog, and at least one fibrinolytic agent, and
packaging comprising instructions for administering the composition to a subject.

28. The kit according to claim 27, wherein the at least one thrombin analog comprises a WE thrombin analog.

29. The kit according to claim 28, wherein the at least one WE thrombin analog comprises the amino acid sequence set forth in SEQ ID NO:2.

30. The kit according to claim 27, wherein the at least one thrombin analog comprises an E-WE thrombin analog.

31. The kit according to claim 30, wherein the at least one E-WE thrombin analog comprises the amino acid sequence set forth in SEQ ID NO:1.

32. The kit according to claim 30, wherein the at least one E-WE thrombin analog comprises the amino acid sequence set forth in SEQ ID NO:22.

33. The kit according to claim 27, wherein the at least one antithrombotic thrombin analog comprises of the amino acid sequence set forth in SEQ ID NO:2 and the at least one fibrinolytic agent comprises tPA.

34. The kit according to claim 27, where in the at least one antithrombotic thrombin analog is comprised amino acid sequence set forth in SEQ ID NO:1 and the at least one fibrinolytic agent comprises tPA.

35. The kit according to claim 27, where in the at least one antithrombotic thrombin analog is comprised amino add sequence set forth in SEQ ID NO:22 and the at least one fibrinolytic agent comprises tPA.

36. The kit according to claim 27, wherein the at least one WE thrombin analog comprises the amino add sequence set forth in SEQ ID NO:2, and the at least one fibrinolytic agent is selected from the group consisting of scuPA, tPA, uPA, tcuPA, streptokinase, rt-PA, alteplase, rt-PA derivatives, reteplase, lanoteplase, TNK-re-PA anisoylated plasminogen streptokinase complex, anistreplase, streptokinase derivative, and combinations thereof.

37. The kit according to claim 27, wherein the at least one E-WE thrombin analog comprises the amino acid sequence set forth in SEC) ID NO:1, and the at least one fibrinoiytic agent is selected from the group consisting of scuPA, tPA, uPA, tcuPA, streptokinase, rt-PA, alteplase, rt-PA derivatives, reteplase, lanoteplase, TNK-re-PA, anisoylated plasminogen streptokinase complex, anistreplase, streptokinase derivative, and combinations thereof.

38. The kit according to claim 27, wherein the at least one E-WE thrombin analog comprises the amino acid sequence set forth in SEQ ID NO:22, and the at least one fibrinolytic agent is selected from the group consisting of scuPA, tPA, uPA, tcuPA, streptokinase, rt-PA, alteplase, rt-PA derivatives, reteplase, lanoteplase, TN -re-PA, anisoylated plasminogen streptokinase complex, anistreplase, streptokinase derivative, and combinations thereof.

39. The kit according to claim 27, further comprising at least one additional pharmaceutically acceptable element, wherein the element is selected from the group consisting of carrier, diluent, excipient, wetting agent, emulsifier, buffer, adjuvant, viscosity additive, preservative, acid, base, salt, sugar, and variations and combinations thereof.


SEQUENCE LISTING (24 sequences)

SEQ ID NO: 1 - protein, 295 aa, Artificial Sequence
  Description of Artificial Sequence Synthetic polypeptide
TFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSAGAGCDRDGKYGFYTHVFRLKKWIQKVIDQFGE

SEQ ID NO: 2 - protein, 295 aa, Artificial Sequence; identical to SEQ ID NO: 1
  Description of Artificial Sequence Synthetic polypeptide

SEQ ID NO: 3 - protein, 295 aa, Artificial Sequence
  Description of Artificial Sequence Synthetic polypeptide
TFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 4 - protein, 622 aa, Artificial Sequence
  Description of Artificial Sequence Synthetic polypeptide
MAHVRGLQLPGCLALAALCSLVHSQHVFLAPQQARSLLQRVRRANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGRTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 5 - protein, 622 aa, Artificial Sequence
  Description of Artificial Sequence Synthetic polypeptide
MAHVRGLQLPGCLALAALCSLVHSQHVFLAPQQARSLLQRVRRANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGRTATSEYQTFFDGRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSAGAGCDRDGKYGFYTHVFRLKKWIQKVIDQFGE

SEQ ID NO: 6 - protein, 308 aa, Artificial Sequence
  Description of Artificial Sequence Synthetic polypeptide
TATSEYQTFFDGRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSAGAGCDRDGKYGFYTHVFRLKKWIQKVIDQFGE

SEQ ID NO: 7 - protein, 622 aa, Artificial Sequence
  Description of Artificial Sequence Synthetic polypeptide
MAHVRGLQLPGCLALAALCSLVHSQHVFLAPQQARSLLQRVRRANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGRTATSEYQTFFDGRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 8 - protein, 308 aa, Artificial Sequence
  Description of Artificial Sequence Synthetic polypeptide
TATSEYQTFFDGRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 9 - protein, 301 aa, Artificial Sequence
  Description of Artificial Sequence Synthetic polypeptide
TATSEYQTFFDGRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 10 - protein, 622 aa, Artificial Sequence
  Description of Artificial Sequence Synthetic polypeptide
MAHVRGLQLPGCLALAALCSLVHSQHVFLAPQQARSLLQRVRRANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGRTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSAGAGCDRDGKYGFYTHVFRLKKWIQKVIDQFGE

SEQ ID NO: 11 - protein, 308 aa, Artificial Sequence
  Description of Artificial Sequence Synthetic polypeptide
TATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSAGAGCDRDGKYGFYTHVFRLKKWIQKVIDQFGE

SEQ ID NO: 12 - protein, 6 aa, Artificial Sequence
  Description of Artificial Sequence Synthetic peptide
FNPRTF

SEQ ID NO: 13 - protein, 6 aa, Artificial Sequence
  Description of Artificial Sequence Synthetic peptide
FDGRTF

SEQ ID NO: 14 - protein, 7 aa, Artificial Sequence
  Description of Artificial Sequence Synthetic peptide
YIDGRIV

SEQ ID NO: 15 - protein, 298 aa, Artificial Sequence
  Description of Artificial Sequence Synthetic polypeptide
DGRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSAGAGCDRDGKYGFYTHVFRLKKWIQKVIDQFGE

SEQ ID NO: 16 - protein, 298 aa, Artificial Sequence
  Description of Artificial Sequence Synthetic polypeptide
EGRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSAGAGCDRDGKYGFYTHVFRLKKWIQKVIDQFGE

SEQ ID NO: 17 - protein, 298 aa, Artificial Sequence
  Description of Artificial Sequence Synthetic polypeptide
DGRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 18 - protein, 298 aa, Artificial Sequence
  Description of Artificial Sequence Synthetic polypeptide
EGRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 19 - protein, 579 aa, Artificial Sequence
  Description of Artificial Sequence Synthetic polypeptide
ANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGRTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSAGAGCDRDGKYGFYTHVFRLKKWIQKVIDQFGE

SEQ ID NO: 20 - protein, 579 aa, Artificial Sequence
  Description of Artificial Sequence Synthetic polypeptide
ANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGRTATSEYQTFFDGRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSAGAGCDRDGKYGFYTHVFRLKKWIQKVIDQFGE

SEQ ID NO: 21 - protein, 579 aa, Artificial Sequence
  Description of Artificial Sequence Synthetic polypeptide
ANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGRTATSEYQTFFEGRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSAGAGCDRDGKYGFYTHVFRLKKWIQKVIDQFGE

SEQ ID NO: 22 - protein, 298 aa, Artificial Sequence; identical to SEQ ID NO: 15
  Description of Artificial Sequence Synthetic polypeptide

SEQ ID NO: 23 - protein, 298 aa, Artificial Sequence; identical to SEQ ID NO: 16
  Description of Artificial Sequence Synthetic polypeptide

SEQ ID NO: 24 - protein, 298 aa, Artificial Sequence; identical to SEQ ID NO: 18
  Description of Artificial Sequence Synthetic polypeptide